(19) United States
(12) Patent Application Publication
(10) Pub. No.: US 2023/0406896 A1
(43) Pub. Date: Dec. 21, 2023

(54) COMPOSITIONS HAVING MULTIPLE RECOMBINANT HUMAN GROWTH FACTORS INCLUDED THEREIN FOR REDUCING SIGNS OF AGING

(71) Applicant: SkinGen international Inc., Toronto (CA)

(72) Inventors: David Bryant, Brereton (GB); Paul Guilbaud, Belleair Bluffs, FL (US)

(21) Appl. No.: 17/912,247

(22) PCT Filed: Mar. 19, 2021
(86) PCT No.: PCT/IB2021/000149

§ 371 Date: Sep. 16, 2022

Related U.S. Application Data
(60) Provisional application No. 62/991,854, filed on Mar. 19, 2020.

(51) Int. Cl. C07K 14/475; A61K 8/73; A61K 8/11; A61K 8/9789; A61Q 19/08; A61K 8/42; A61K 8/06; A61K 8/66

(52) U.S. Cl. CPC C07K 14/475; A61K 8/735; A61K 8/11; A61K 8/66; A61Q 19/08; A61K 8/42; A61K 8/062; A61K 8/9789

(57) ABSTRACT

Compositions for topical or transdermal use having multiple recombinant human growth factors thereinfor repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin. The composition includes water, and at least five but no more than twelve recombinant human growth factors selected from sh-Polypeptide-1, sh-Polypeptide-11, sh-Polypeptide-31, sh-Oligopeptide-2, sh-Polypeptide-10, sh-Polypeptide-5, sh-Polypeptide-8, sh-Polypeptide-3, sh-Polypeptide-62, Acetyl Octapeptide-17 Amide, sh-oligopeptide-1, sh-Polypeptide-4, and Acetyl sh-Oligopeptide-77 Amide in which, each growth factor, when present in the composition, is present at a concentration ranging from 0.001 wt % to 1 wt % of the overall composition.

### REFERENCE TO AN ELECTRONIC SEQUENCE LISTING

The contents of the electronic sequence listing (305406_241405_SkinGenSequenceListing_ST25.txt; Size: 16.187 bytes; and Date of Creation: Sep. 16, 2022) is herein incorporated by reference in its entirety.

### TECHNICAL FIELD

The present invention relates generally to the field of skin care and aesthetics, and more particularly to compositions for topical and/or transdermal use having multiple recombinant human growth factors and hyaluronic acid included therein for repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin.

### BACKGROUND

Fine lines and wrinkles are often attributed to the loss of collagen and elastin proteins (e.g., proteins comprising the extracellular matrix (ECM)) present in the dermal layers resulting in breakdown of skin thickness and resiliency over time. The loss of collagen and ECM can be generally attributed to multiple factors including biological aging and/or exposure to various environmental factors such as UV, which may further result in photo-aging.

There are invasive and non-invasive techniques for attempting to mitigate signs of aging (fine lines, wrinkles, increased pore diameter, roughened facial texture). For example, the most common surgical interventions available for treatment of facial wrinkles include face-lifts, laser surgery, and injection therapies, including for example, dermal fillers (having crosslinked hyaluronic acid matrices) and/or BOTOX®. Although invasive surgical methods and techniques are a viable option for anti-aging, these treatments are often very painful, must be periodically repeated, and if performed improperly, have detrimental aesthetic and health complications.

In addition to the above mentioned invasive techniques, non-invasive techniques include application of topical formulations consisting of many different ingredients including, alpha/beta hydroxy, retinoic acids, and vitamins and various plant extracts. However, none of these methods are very effective in eliminating wrinkles, and often require multiple, expensive treatments. These products focus on short term skin ‘plumping’ or on having a short term effect rather than looking at helping with improving the underlying cause of the skin aging. Furthermore, some topical formulations act as skin irritants (often resulting in red, inflamed skin and/or dry skin), to elicit wound healing responses, but do not successfully replenish the thinning skin with adequate proteins for treatment and/or prevention of age-related defects. In view of the above mentioned pitfalls, U.S. Pat. No. 8,518,879 provided formulations having a conditioned medium derived from transformed eulkaryotic cells that included an exosomally secreted growth factor. However, it should be noted that formulations disclosed in U.S. Pat. No. 8,518,879 suffer from many deficiencies including potentially inducing immunogenic responses in users while also being susceptible to growth factor inactivation. Moreover, and due to constitutive transformation of the eukaryotic cells disclosed in U.S. Pat. No. 8,518,879, quality control is a major issue due to the eukaryotic cell lines potentially being mosaics, resulting in varying concentrations of the desired actives being produced as well as potentially producing spontaneous mutations/mutants resulting during cell culture of its eukaryotic cells leading to highly variable lots within its conditioned medium.

Thus, in view of the above, additional anti-aging techniques, compositions, and/or formulations are needed.

### SUMMARY

It is an object to provide topical and/or transdermal compositions/formulations having multiple recombinant human growth factors therein for repairing and/or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin while concurrently avoiding and/or minimizing the problems observed with current invasive and non-invasive techniques and the inability of the body to produce the Growth Factors in endogenous amounts that adequately stimulate the cell regeneration due to the aging process. In particular, these compositions/formulations utilize multiple nano-encapsulated, recombinant human growth factors, for effective delivery, repair, and regeneration of aging skin as well as hair. These compositions may be further used in combination with other treatments and/or protocols (e.g., micro-needling protocols) to maximize aesthetic efficacy and outcome for the patient. The combination of multiple growth factors (recombinant human growth factors) as disclosed herein has a more proliferative effect on the production of cellular intermediaries needed for skin rejuvenation than, for example, formulations having a single growth factor. In particular, when combinations of multiple recombinant human growth factors are used, as disclosed herein, at therapeutic doses, they advantageously initiate skin repair and regeneration processes without inducing immunogenic responses. In certain aspects, the combination of recombinant human growth factors disclosed herein work synergistically to enhance their individual effects.

In certain aspects, disclosed is a composition for topical or transdermal use having multiple recombinant human growth factors therein for repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin, the composition comprising: (a) water at a concentration ranging from 55 wt % to 85 wt % of the overall composition; (b) optionally, and when present, hyaluronic acid or an acceptable salt thereof at a concentration ranging from 0.5 wt % to 6 wt % of the overall composition with a molecular weight ranging from 150 kDA to 600 kDA (and preferably uncrosslinked); and (c) at least 5 but no more than 12 recombinant human growth factors selected from sh-Polypeptide-1 (SEQ ID NO 2), sh-Polypeptide-11 (SEQ ID NO 3), sh-Polypeptide-31 (SEQ ID NO 4), sh-Oligopeptide-2 (SEQ ID NO 6), sh-Polypeptide-10 (SEQ ID NO 7), sh-Polypeptide-5 (SEQ ID NO 8), sh-Polypeptide-8 (SEQ ID NO 9), sh-Polypeptide-3 (SEQ ID NO 10), sh-Polypeptide-62 (SEQ ID NO 1), Acetyl Octapeptide-17 Amide (SEQ ID NO 5), sh-oligopeptide-1 (SEQ ID NO 11), sh-Polypeptide-4 (SEQ ID NO 12), and Acetyl sh-Oligopeptide-77 Amide (SEQ ID NO 13) in which, each growth factor, when present in the composition, is present at a concentration ranging from 0.001 wt % to 1 wt % of the overall composition. To minimize and/or avoid any unwanted immunogenic response from the user while further maximizing quality control, the recombinant human growth factors are not derived from transformed eukaryotic cells and do not include conditioned media/medium but are instead derived from transgenic bacteria and are subsequently isolated/purified therefrom. Regarding each of the compositions/formulations mentioned immediately below, each composition/formulation includes the above mentioned water, hyaluronic acid, and varying numbers of recombinant human growth factors. In certain aspects, the recombinant human growth factors of the composition are sh-Polypeptide-1 is SEQ ID NO 2, sh-Polypeptide-11 is SEQ ID NO 3, sh-Polypeptide-31 is SEQ ID NO 4, shOligopeptide-2 is SEQ ID NO 6, the sh-Polypeptide-10 is SEQ ID NO 7, sh-Polypeptide-5 is SEQ ID NO 8, sh-Polypeptide-8 is SEQ ID NO 9, sh-Polypeptide-3 is SEQ ID NO 10, sh-Polypeptide-62 is SEQ ID NO 1, Acetyl Octapeptide-17 Amide is SEQ ID NO 5, sh-oligopeptide-1 is SEQ ID NO 11, sh-Polypeptide-4 is SEQ ID NO 12, and Acetyl sh-Oligopeptide-77 Amide is SEQ ID NO 13. In other aspects, the recombinant human growth factors of the composition are when sh-Polypeptide-1 comprises a sequence at least 90%, 95%, or 98% identical to SEQ ID NO 2, the sh-Polypeptide-11 comprises a sequence at least 90%, 95%, or 98% identical to SEQ ID NO 3, the sh-Polypeptide-31 comprises a sequence at least 90%, 95%, or 98% identical to SEQ ID NO 4, the shOligopeptide-2 comprises a sequence at least 90%, 95%, or 98% identical to SEQ ID NO 6, the sh-Polypeptide-10 comprises a sequence at least 90%, 95%, or 98% identical to SEQ ID NO 7, the sh-Polypeptide-5 comprises a sequence at least 90%, 95%, or 98% identical to SEQ ID NO 8, the sh-Polypeptide-8 comprises a sequence at least 90%, 95%, or 98% identical to SEQ ID NO 9, the sh-Polypeptide-3 comprises a sequence at least 90%, 95%, or 98% identical to SEQ ID NO 10, the sh-Polypeptide-62 comprises a sequence at least 90%, 95%, or 98% identical to SEQ ID NO 1, Acetyl Octapeptide-17 Amide comprises a sequence at least 90%, 95%, or 98% identical to SEQ ID NO 5, the sh-oligopeptide-1 comprises a sequence at least 90%, 95%, or 98% identical to SEQ ID NO 11, the sh-Polypeptide-4 comprises a sequence at least 90%, 95%, or 98% identical to SEQ ID NO 12, and the Acetyl sh-Oligopeptide-77 Amide comprises a sequence at least 90%, 95%, or 98% identical to SEQ ID NO 13.

In certain aspects, the composition is an aqueous solution for topical use comprising either alone or in conjunction with microneedling (with microneedling occurring either pre-application or post-application of the composition) in which the composition includes water (as mentioned above) and at least 8 growth factors are present, hyaluronic acid at a concentration ranging from 1.5 wt % to 4.5 wt % of the overall composition; and further comprises: (i) a nanoencapsulating agent that encapsulates the recombinant human growth factors and present in the composition at an effective amount to deliver the recombinant human growth factors to cells, keratinous materials, and/or the extracellular matrix in a human, (ii) a Swertia chirata extract present at an effective amount for stimulating endogenous keratinocyte growth factor production to induce keratinocyte proliferation and epidermis regeneration to increase skin volume and/or reduce cutaneous signs of aging, (iii) an emollient, and (iv) a skin protecting agent present at an effective amount to stimulate endogenous production of collagen VII, laminin-5, and/or fibronectin. In this aspect, the Swertia chirata extract is present at a concentration ranging from 1 wt % to 8 wt % of the overall composition. In this aspect, the skin protecting agent is present at a concentration ranging from 1 wt % to 5 wt % of the overall composition. In this aspect, the skin protecting agent comprises a combination of glycerin, water, dextran, and caproyl tetrapeptide-3. In this aspect, the composition further comprises a water soluble skin conditioning agent configured to minimize enlarged pores, Lighten lax pores, improve uneven skin tone, soften fine lines and wrinkles, diminish dullness, and/or strengthen a weakened skin surface. In this aspect, the water soluble skin conditioning agent is present at a concentration ranging from 0.1 wt % to 5 wt % of the overall composition. In this aspect, the water soluble skin conditioning agent is niacinamide. In this aspect, the nanoencapsulating agent is a C1-C6 alkylene glycol (e.g., either a diol or a polyol) suitable for topical and/or transdermal use and lecithin mixture present at a ratio ranging from 4:1 to 2.3:1 of C1-C6 alkylene glycol to lecithin, and in certain aspects, the a C1-C6 alkylene glycol suitable for topical and/or transdermal use is present at a concentration of 70 wt % to 80 wt % of the overall concentration of the nanoencapsulating agent and lecithin is present at a concentration of 20 to 30 wt % of the overall concentration of the nanoencapsulating agent. In certain aspects, the C1-C6 alkylene glycol is propanediol and lecithin is a soybean (G. max) extract (or alternatively a plurality of isolates that are combined with one another) in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanoloamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent.

In additional aspects, the composition is an oil in water emulsion for topical use and transdermal absorption either alone or in conjunction with microneedling (with microneedling occurring either pre-application or post-application of the composition), the composition comprising: water (as mentioned above) and at least 8 recombinant human growth factors are present, and hyaluronic acid or an acceptable salt thereof at a concentration ranging from 0.5 wt % to 6 wt % of the overall composition with a molecular weight ranging from 150 kDA to 600 kDA (and preferably uncrosslinked), and further comprises: (i) a nanoencapsulating agent that encapsulates the recombinant human growth factors and present in an effective amount to deliver the recombinant human growth factors to cells, keratinous materials, and/or the extracellular matrix in a human; (ii) a plurality of anti-inflammatory agents and/or antioxidants at a concentration ranging from 1 wt % to 7 wt % of the overall composition; (iii) a combination of liposomally encapuslated bacterial derived photolyase(s), plant-derived roxisome(s), and bacterial derived endonuclease(s) that are present in an effective amount to prevent and/or reduce photoaging associated with ultraviolet (UV) exposure by enhancing endogenous DNA repair. In certain aspects, the growth factors and combination of photolyases, plant-derived roxisomes, and bacterial derived endonuclease improve skin after sun damage by initiating the repair and regenerative processes and help speed up the rate of improvement of the main signs of skin aging. In this aspect, the combination of liposomally encapuslated bacterial derived photolyase(s), plant-derived roxisome(s), and bacterial derived endonucleases are present at a concentration ranging from 0.3 wt % to 10 wt % of the overall composition. In this aspect, bacterial derived photolyases are present at a concentration ranging from 0.1 wt % to 3.5 wt % of the overall composition. In this aspect, the bacterial derived photolyase is a cyanobacteria photolyase. In this aspect, the cyanobacteria photolyase is an Anacystis nidulans photolyase. In this aspect, the plant derived roxisome(s) are present at a concentration ranging from 0.1 wt % to 3.5 wt % of the overall composition. In this aspect, the plant derived roxisome(s) is 8-oxo-guanine glycosylase from A. thaliana. In this aspect, the bacterial derived endonucleases are present at a concentration ranging from 0.1 wt % to 3.5 wt % of the overall composition. In this aspect, the bacterial derived endonuclease(s) are bacterial endonucleases from M. Luteus. In this aspect, the nanoencapsulating agent is a C1-C6 alkylene glycol suitable for topical and/or transdermal use and lecithin mixture present at a ratio ranging from 4:1 to 2.3:1 of C1-C6 alkylene glycol to lecithin, and in certain aspects, the a C1-C6 alkylene glycol suitable for topical and/or transdermal use is present at a concentration of 70 wt % to 80 wt % of the overall concentration of the nanoencapsulating agent and lecithin is present at a concentration of 20 to 30 wt % of the overall concentration of the nanoencapsulating agent. In certain aspects, the C1-C6 alkylene glycol is propandiol and lecithin is a soybean (G. max) extract (or alternatively a plurality of isolates that are combined with one another) in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanoloamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent. In this aspect, the composition further comprising an emollient and an emulsifier present at a concentration ranging from 6 wt % to 32 wt % of the overall composition. In this aspect, retinol is present as an antioxidant in the composition at a concentration ranging from 1 wt % to 3 wt %. In this aspect, the liposomes encapsulating bacterial derived photolyase(s), plant-derived roxisome(s), and bacterial derived endonucleases are comprised of lecithin, and the lecithin is soybean lecithin.

In yet another aspect, the composition is an aqueous solution for topical use on a human scalp either alone or in conjunction with microneedling (with microneedling occurring either pre-application or post-application of the composition), the composition comprising water and hyaluronic acid (as mentioned above) and at least 10 recombinant human growth factors, and a nanoencapsulating agent that encapsulates the recombinant human growth factors and present in an effective amount to deliver the recombinant human growth factors to the human scalp to induce hair growth and/or re-growth and/or rejuvenate scalp appearance In certain aspects, the nanoencapsulating agent includes a C1-C6 alkylene glycol suitable for topical and/or transdermal use and lecithin mixture present at a ratio ranging from 4:1 to 2.3:1 of C1-C6 alkylene glycol to lecithin, and in certain aspects, the a C1-C6 alkylene glycol suitable for topical and/or transdermal use is present at a concentration of 70 wt % to 80 wt % of the overall concentration of the nanoencapsulating agent and lecithin is present at a concentration of 20 to 30 wt % of the overall concentration of the nanoencapsulating agent. In certain aspects, the C1-C6 alkylene glycol is propanediol and lecithin is a soybean (G. max) extract (or alternatively a plurality of isolates that are combined with one another) in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanoloamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent.

Also disclosed is a kit including at least one of the compositions mentioned above that is packaged within a container. The kit further includes a plurality of sterile microneedles packaged within the kit and configured to enhance transdermal delivery of the composition post-application of the composition to the human cells and/or keratinous materials by creating punctures (puncturing) in a user's stratum corneum to induce a wound healing response and to enhance delivery to improve any one of skin surface appearance, cutaneous signs of chronological aging and/or aging signs induced by external factors such as prolonged exposure to ultraviolet (UV) exposure, and/or impaired surface appearance of the skin.

Also disclosed is a method of reducing wrinkles and/or fine lines over a predetermined time period, the method comprising: (a) applying on the skin of a subject at least one of the compositions disclosed above; and (b) repeating the application of the composition to the skin of the subject at predetermined time periods thereby reducing wrinkles, fine lines, and/or the appearance thereof during the predetermined time period. In certain aspects, this method further comprises: before step (a) performing a microneedling procedure on the subject to increase delivery efficacy of the composition to the subject. In certain aspects, this method further comprises after step (a) and before step (b) performing a microneedling procedure on the subject to increase delivery efficacy of the composition to the subject. In certain aspects, this method further comprises after each application of the composition to the skin of the subject performing a microneedling procedure on the subject to increase delivery efficacy of the composition to the subject.

Also disclose is a method for reducing pore size over a predetermined time period, the method comprising: (a) applying on the skin of a subject at least one of the compositions disclosed above; and (b) repeating the application of the composition to the skin of the subject at predetermined time periods thereby reducing pore size and/or the appearance thereof during the predetermined time period. In certain aspects, this method further comprises before step (a) performing a microneedling procedure on the subject to increase delivery efficacy of the composition to the subject. In certain aspects, this method further comprises after step (a) and before step (b) performing a microneedling procedure on the subject to increase delivery efficacy of the composition to the subject. In certain aspects, this method further comprises after each application of the composition to the skin of the subject performing a microneedling procedure on the subject to increase delivery efficacy of the composition to the subject.

Also disclosed is a method of enhancing skin texture over a predetermined time period, the method comprising: (a) applying on the skin of a subject at least one of the compositions disclosed above; and (b) repeating the application of the composition to the skin of the subject at predetermined time periods thereby improving and/or enhancing skin texture and/or the appearance thereof during the predetermined time period. In certain aspects, this method further comprises before step (a) performing a microneedling procedure on the subject to increase delivery efficacy of the composition to the subject. In certain aspects, this method further comprises after step (a) and before step (b) performing a microneedling procedure on the subject to increase delivery efficacy of the composition to the subject. In certain aspects, this method further comprises after each application of the composition to the skin of the subject performing a microneedling procedure on the subject to increase delivery efficacy of the composition to the subject.

Embodiments of the invention can include one or more or any combination of the above features and configurations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 depicts an exemplary transgenic construct used to generate the recombinant human growth factors disclosed herein;

FIG. 2 is an Optical Density (O.D.) growth curve depicting eukaryotic cell proliferation when treated with a formulation having a single recombinant human growth factor (i.e., epidermal growth factor (EGF));

FIG. 3 is an Optical Density (O.D.) growth curve depicting eukaryotic cell proliferation when treated with an exemplary formulation as disclosed herein having ten recombinant human growth factors during the same time period as that shown in FIG. 2;

FIG. 4 is a graph merging the data from FIGS. 2 and 3 further evidencing the enhanced proliferative effect of an exemplary composition as disclosed herein having ten recombinant human growth factors;

FIG. 5 depicts an exemplary microneedle for use in conjunction with the composition(s) as disclosed herein;

FIG. 6a depicts the scoring parameters/categories for scoring/grading fine lines and wrinkles on the subject's face during the dermatological assessment while using an exemplary composition as disclosed herein; FIG. 6b is a table depicting the statistical data compiled over the twelve week testing period; and FIG. 6c is a graph depicting the mean values and improvement of the subject's fine lines and wrinkles during the dermatological assessment over the twelve week period while using an exemplary composition as disclosed herein;

FIG. 7a depicts the scoring parameters/categories for scoring/grading evenness of the subject's skin tone during the dermatological assessment while using an exemplary composition as disclosed herein; FIG. 7b is a table depicting the statistical data compiled over the twelve week testing period; and FIG. 7c is a graph depicting the mean values and improvement of the evenness of the subject's skin tone during the dermatological assessment over the twelve week period while using an exemplary composition as disclosed herein;

FIG. 8a depicts the scoring parameters/categories for scoring/grading evenness of the subject's skin clarity during the dermatological assessment while using an exemplary composition as disclosed herein; FIG. 8b is a table depicting the statistical data compiled over the twelve week testing period; and FIG. 8c is a graph depicting the mean values and improvement of the subject's skin clarity during the dermatological assessment over the twelve week period while using an exemplary composition as disclosed herein;

FIG. 9a depicts the scoring parameters/categories for scoring/grading the subject's age-spots/hyper-pigmentation during the dermatological assessment while using an exemplary composition as disclosed herein; FIG. 9b is a table depicting the statistical data compiled over the twelve week testing period; and FIG. 9c is a graph depicting the mean values and improvement of the subject's age-spots/hyper pigmentation during the dermatological assessment over the twelve week period while using an exemplary composition as disclosed herein;

FIG. 10a depicts the scoring parameters/categories for scoring/grading the subject's skin smoothness during the dermatological assessment while using an exemplary composition as disclosed herein; FIG. 10b is a table depicting the statistical data compiled over the twelve week testing period; and FIG. 10c is a graph depicting the mean values and improvement of the subject's skin smoothness during the dermatological assessment over the twelve week period while using an exemplary composition as disclosed herein;

FIG. 11a depicts the scoring parameters/categories for scoring/grading the subject's skin firmness/laxity during the dermatological assessment while using an exemplary composition as disclosed herein; FIG. 11b is a table depicting the statistical data compiled over the twelve week testing period; and FIG. 11c is a graph depicting the mean values and improvement of the subject's skin firmness/laxity during the dermatological assessment over the twelve week period while using an exemplary composition as disclosed herein;

FIG. 12a depicts the scoring parameters/categories for scoring/grading the subject's skin moisturization/hydration during the dermatological assessment while using an exemplary composition as disclosed herein; FIG. 12b is a table depicting the statistical data compiled over the twelve week testing period; and FIG. 12c is a graph depicting the mean values and improvement of the subject's skin moisturization/hydration during the dermatological assessment over the twelve week period while using an exemplary composition as disclosed herein;

FIG. 13a depicts statistical data from the 3D imaging (Antera software) and wrinkle assessment of the subject's forehead (subjects having fine, mild, and deep wrinkles respectively) over the twelve week testing period while using an exemplary composition as disclosed herein; and FIG. 13b is a graph compiled based on the 3D imaging (Antera software) depicting the mean values and improvement of the subject's forehead wrinkles (subjects having fine, mild, and deep wrinkles respectively) over the twelve week period while using an exemplary composition as disclosed herein;

FIG. 14a is a photograph (3D imaging (Antera software)) showing a subject during week 1 having fine forehead wrinkles; FIG. 14b is a photograph (3D imaging (Antera software)) showing improvement of the forehead wrinkles in the same subject (as shown in FIG. 14a) after twelve weeks of treatment with the exemplary composition as disclosed herein; FIG. 14c is a photograph (3D imaging (Antera software)) showing a subject during week 1 having mild/medium forehead wrinkles; FIG. 14d is a photograph (3D imaging (Antera software)) showing improvement of the forehead wrinkles in the same subject (as shown in FIG. 14c) after twelve weeks of treatment with the exemplary composition as disclosed herein; FIG. 14e is a photograph (3D imaging (Antera software)) showing a subject during week 1 having deep forehead wrinkles; and FIG. 14f is a photograph (3D imaging (Antera software)) showing improvement of the forehead wrinkles in the same subject (as shown in FIG. 14e) after twelve weeks of treatment with the exemplary composition as disclosed herein;

FIG. 15a depicts statistical data from the 3D imaging (Antera software) and wrinkle assessment of the subject's crow's feet (subjects having fine, mild, and deep wrinkles respectively) over the twelve week testing period while using an exemplary composition as disclosed herein; and FIG. 15b is a graph compiled based on the 3D imaging (Antera software) depicting the mean values and improvement of the subject's crow's feet (subjects having fine, mild, and deep wrinkles respectively) over the twelve week period while using an exemplary composition as disclosed herein;

FIG. 16a is a photograph (3D imaging (Antera software)) showing a subject during week 1 having crow's feet (fine wrinkles); FIG. 16b is a photograph (3D imaging (Antera software)) showing improvement of the crow's feet in the same subject (as shown in FIG. 16a) after twelve weeks of treatment with the exemplary composition as disclosed herein; FIG. 16c is a photograph (3D imaging (Antera software)) showing a subject during week 1 having mild/medium crow's feet (mild/medium wrinkles); FIG. 16d is a photograph (3D imaging (Antera software)) showing improvement of the crow's feet in the same subject (as shown in FIG. 16c) after twelve weeks of treatment with the exemplary composition as disclosed herein; FIG. 16e is a photograph (3D imaging (Antera software)) showing a subject during week 1 having deep crow's feet (deep wrinkles); and FIG. 16f is a photograph (3D imaging (Antera software)) showing improvement of the crow's feet in the same subject (as shown in FIG. 16e) after twelve weeks of treatment with the exemplary composition as disclosed herein;

FIG. 17a depicts statistical data from the 3D imaging (Antera software) and wrinkle assessment of the subject's nasolabial folds (subjects having fine, mild, and deep wrinkles respectively) over the twelve week testing period while using an exemplary composition as disclosed herein; and FIG. 17b is a graph compiled based on the 3D imaging (Antera software) depicting the mean values and improvement of the wrinkles in the subject's nasolabial folds (subjects having fine, mild, and deep wrinkles respectively) over the twelve week period while using an exemplary composition as disclosed herein;

FIG. 18a is a photograph (3D imaging (Antera software)) showing a subject during week 1 having fine wrinkles in the nasolabial folds; FIG. 18b is a photograph (3D imaging (Antera software)) showing improvement of the nasolabial fold wrinkles in the same subject (as shown in FIG. 18a) after twelve weeks of treatment with the exemplary composition as disclosed herein; FIG. 18c is a photograph (3D imaging (Antera software)) showing a subject during week 1 having mild/moderate wrinkles in the nasolabial folds; FIG. 18d is a photograph (3D imaging (Antera software)) showing improvement of the nasolabial fold wrinkles in the same subject (as shown in FIG. 18c) after twelve weeks of treatment with the exemplary composition as disclosed herein; FIG. 18e is a photograph (3D imaging (Antera software)) showing a subject during week 1 having deep wrinkles in the nasolabial folds; and FIG. 18f is a photograph (3D imaging (Antera software)) showing improvement of the nasolabial fold wrinkles in the same subject (as shown in FIG. 18e) after twelve weeks of treatment with the exemplary composition as disclosed herein;

FIG. 19a depicts statistical data from the 3D imaging (Antera software) and texture assessment of the subject's forehead (subjects having fine, mild, and deep wrinkles respectively) over the twelve week testing period while using an exemplary composition as disclosed herein; and FIG. 19b is a graph compiled based on the 3D imaging (Antera software) depicting the mean values and improvement of the texture in the subject's forehead (subjects having fine, mild, and deep wrinkles respectively) over the twelve week period while using an exemplary composition as disclosed herein;

FIG. 20a is a photograph (3D imaging (Antera software)) showing a subject during week 1 having fine forehead texture; FIG. 20b is a photograph (3D imaging (Antera software)) showing improvement of forehead texture in the same subject (as shown in FIG. 20a) after twelve weeks of treatment with the exemplary composition as disclosed herein; FIG. 20c is a photograph (3D imaging (Antera software)) showing a subject during week 1 having mild/moderate forehead texture; FIG. 20d depicts is a photograph (3D imaging (Antera software)) showing improvement of forehead texture in the same subject (as shown in FIG. 20c) after twelve weeks of treatment with the exemplary composition as disclosed herein; FIG. 20e is a photograph (3D imaging (Antera software)) showing a subject during week 1 having deep forehead texture; and FIG. 20f is a photograph (3D imaging (Antera software)) showing improvement of forehead texture in the same subject (as shown in FIG. 20e) after twelve weeks of treatment with the exemplary composition as disclosed herein;

FIG. 21a depicts statistical data from the 3D imaging (Antera software) and texture assessment of the subject's crow's feet (subjects having fine, mild, and deep wrinkles respectively) over the twelve week testing period while using an exemplary composition as disclosed herein; and FIG. 21b is a graph compiled based on the 3D imaging (Antera software) depicting the mean values and improvement of the texture in the subject's crow feet texture (subjects having fine, mild, and deep wrinkles respectively) over the twelve week period while using an exemplary composition as disclosed herein;

FIG. 22a is a photograph (3D imaging (Antera software)) showing a subject during week 1 having fine crow's feet texture; FIG. 22b is a photograph (3D imaging (Antera software)) showing improvement of crow's feet texture in the same subject (as shown in FIG. 22a) after twelve weeks of treatment with the exemplary composition as disclosed herein; FIG. 22c is a photograph (3D imaging (Antera software)) showing a subject during week 1 having mild/moderate crow's feet texture; FIG. 22d is a photograph (3D imaging (Antera software)) showing improvement of crow's feet texture in the same subject (as shown in FIG. 22c) after twelve weeks of treatment with the exemplary composition as disclosed herein; FIG. 22e is a photograph (3D imaging (Antera software)) showing a subject during week 1 having deep crow's feet texture; and FIG. 22f is a photograph (3D imaging (Antera software)) showing improvement of crow's feet texture in the same subject (as shown in FIG. 22e) after twelve weeks of treatment with the exemplary composition as disclosed herein;

FIG. 23a depicts statistical data from the 3D imaging (Antera software) and texture assessment of the subject's nasolabial folds (subjects having fine, mild, and deep wrinkles respectively) over the twelve week testing period while using an exemplary composition as disclosed herein; and FIG. 23b is a graph compiled based on the 3D imaging (Antera software) depicting the mean values and improvement of the texture in the subject's nasolabial fold texture (subjects having fine, mild, and deep wrinkles respectively) over the twelve week period while using an exemplary composition as disclosed herein;

FIG. 24a is a photograph (3D imaging (Antera software)) showing a subject during week 1 having fine nasolabial texture; FIG. 24b is a photograph (3D imaging (Antera software)) showing improvement of nasolabial texture in the same subject (as shown in FIG. 24a) after twelve weeks of treatment with the exemplary composition as disclosed herein; FIG. 24c is a photograph (3D imaging (Antera software)) showing a subject during week 1 having mild/moderate nasolabial texture; FIG. 24d is a photograph (3D imaging (Antera software)) showing improvement of nasolabial texture in the same subject (as shown in FIG. 24c) after twelve weeks of treatment with the exemplary composition as disclosed herein; FIG. 24e is a photograph (3D imaging (Antera software)) showing a subject during week 1 having deep nasolabial texture; and FIG. 24f is a photograph (3D imaging (Antera software)) showing improvement of nasolabial texture in the same subject (as shown in FIG. 24e) after twelve weeks of treatment with the exemplary composition as disclosed herein;

FIG. 25a depicts statistical data from the CR imaging (Visia software) assessing wrinkle spread in the subjects over the twelve week testing period while using an exemplary composition as disclosed herein; FIG. 25b is a graph compiled based on the CR imaging (Visia software) depicting the mean values of decreased wrinkle spread in the subjects over the twelve week period while using an exemplary composition as disclosed herein; FIG. 25c shows a subject at V2 (week 1); and FIG. 25d shows the same subject at V6 (week 8);

FIG. 26a depicts statistical data from the CR imaging (Visia software) assessing texture in the subjects over the twelve week testing period while using an exemplary composition as disclosed herein; FIG. 26b is a graph compiled based on the CR imaging (Visia software) depicting the mean values of subject's skin texture over the twelve week period while using an exemplary composition as disclosed herein; FIG. 26c shows a skin texture of a subject at V2 (week 1); and FIG. 26d shows the skin texture of the same subject at V6 (week 8);

FIG. 27a depicts statistical data from the CR imaging (Visia software) assessing pore spread in the subjects over the twelve week testing period while using an exemplary composition as disclosed herein s; FIG. 27b is a graph compiled based on the CR imaging (Visia software) depicting the mean values of decreased pore spread in the subjects over the twelve week period while using an exemplary composition as disclosed herein; FIG. 27c shows the pores of a subject at V2 (week 1); and FIG. 27d shows the pores of the same subject at V6 (week 8);

FIG. 28 is statistical data compiled from image based comparative assessments of various time points (weeks 1, 4, 8, and 12) for the subjects assessing the subjects' crow's feet, nasolabal folds, frown lines, age spots, evenness of skin, and skin texture;

FIG. 29a is a photograph depicting a subject's crow's feet at week 2 (V2); and FIG. 29b shows the same subject's crow's feet at week 12 (V8) having markedly reduced crow's feet;

FIG. 30a is a photograph depicting a subject's nasolabial folds at week 2 (V2); FIG. 30b shows the same subject's nasolabial folds at week 12 (V8) having markedly reduced wrinkling; and

FIG. 31a is a photograph depicting a subject's frown lines at week 2 (V2); and FIG. 31b shows the same subject's frown lines at week 12 (V8) having markedly reduced wrinkling.

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

“Optional” or “optionally” means that the subsequently described event or circumstance can or cannot occur, and that the description includes instances where the event or circumstance occurs and instances where it does not.

As used herein, the term “about” is used to provide flexibility to a numerical range endpoint by providing that a given value may be “slightly above” or “slightly below” the endpoint without affecting the desired result.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

The compositions and methods described herein can comprise, consist of, or consist essentially of the essential elements and limitations described herein, as well as any additional or optional ingredients, components, or limitations described herein.

The term sterile, relates to an environment ensuring the safety required for preparing a composition which can be safely used by topical administration on damaged skin surfaces and/or relates to a composition which is prepared in a sterile environment or made sterile with a sterilization method which may be chosen among the ones known by the one skilled in the art. Indeed, for obvious reasons, it is essential that the compositions disclosed herein are devoid of any contaminant capable of initiating undesirable reactions and/or side effects and are initially sterile (and/or remain sterile while packaged within vials and/or containers).

The term topical refers to a composition which is intended to be applied on the skin surface (dermis) of a subject.

The term transdermal means absorbed and/or to be absorbed through a subjects epidermis and/or dermal layers.

The phrase “effective amount” as used herein relates to the amount/concentration used within the disclosed composition(s) in order to achieve the desired/specified effects.

The term/phrase “microneedling or micro-needling” relates to the puncture of the skin to various depths with very fine needles. This procedure causes a controlled injury inducing a controlled wound response in which the skin synthetizes more collagen thus having an anti-aging effect and/or improving the skin/skin appearance of the subject.

“Peptide” or “peptide sequence” may be used to refer to a natural or synthetic molecule comprising two or more amino acids linked by the carboxyl group of one amino acid to the alpha amino group of another. The peptide is not limited by length, and thus “peptide” can include a peptide fragment, a polypeptide(s), and full-length proteins.

When describing variants in proteins, polypeptides, or peptides, the term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 65%, 70%, 75%, 80%, 85%, 90%, 95%, 96%, 97%, 98%, or 99% homology to a reference sequence.

The terms “homology,” “identity or identical,” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

It is understood that any given particular aspect of the disclosed compositions and methods can be easily compared to the specific examples and embodiments disclosed herein. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

General Formulation/Composition

Disclosed immediately below is a general formulation and/or composition that includes chemical components and growth factors that are included within each of the specific, individual compositions/formulations disclosed in detail further below.

Each of the formulations/compositions disclosed herein generally directed to topical or transdermal use. Each composition/formulation includes multiple recombinant human growth factors (shown in Table 1 below) therein for repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin.

Each of these formulations are preferably water based and have water at a concentration ranging from 55 wt % to 85 wt % of the overall composition. Water concentration may be varied according to each individual composition disclosed herein, and water concentration may fall within any range of the above disclosed endpoints.

In addition, the formulations disclosed herein may include hyaluronic acid that, when included, aids in enhancing skin hydration, elasticity, and ECM repair and/or maintenance when used in an effective amount within the composition(s)/formulation(s) disclosed herein. It should be noted that hyaluronic acid (CAS No.: 9067-32-7) as disclose herein also refers to physiological acceptable salts thereof as well; these physiologically acceptable salts include sodium salt, the potassium salt, the zinc salt and the silver salt, its derivatives and mixture thereof. In certain aspects, the hyaluronic acid is preferably a low molecular weight hyaluronic acid ranging from 150 kDA to 600 kDA at a concentration ranging from 0.5 wt % to 6 wt % of the overall composition, more preferably from 1 wt % to 5 wt % the overall composition. Lower weight hyaluronic acid is preferred for the transdermal and topical applications disclosed herein. As hyaluronic acid molecular weight increases, the more difficult transdermal delivery of the hyaluronic acid becomes, which may further affect the composition efficacy and desired results (i.e, repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin.) disclosed herein. The hyaluronic acid disclosed herein may be uncrosslinked (non-crosslinked), crosslinked, or a combination thereof. In certain aspects, each formulation includes only uncrosslinked (non-crosslinked) hyaluronic acid, which is more easily transdermally and/or topically applied and delivered to achieve the desired effects disclose herein than crosslinked hyaluronic acid. However, in alternative aspects, the composition includes mixtures or crosslinked and uncrosslinked hyaluronic acid. The hyaluronic acid as disclosed herein may in certain aspects bind to the cell surface receptor CD 44 activating an endogenous keratinocyte proliferation cascade and up regulation of endogenous hyaluronic acid production via this keratinocyte cascade, which ultimately resulting in thickening of the epidermis and strengthening of the ECM.

In certain aspects and to further increase delivery of the above mentioned hyaluronic acid and when present in the compositions, the hyaluronic acid is preferably admixed with a silanol and more preferably an organosilanol for delivery and transdermal and topical absorption purposes. In particular, the silanol and/or organosilanol is methylsilanetriol (CAS No.: 4253-34-3 or CAS No. 2445-53-6), which further aids in miscibility and bioavailability of the hyaluronic acid within the compositions/formulations disclosed herein. In this aspect, the organosilanetriol (CAS No.: 4253-34-3 or methylsilanetriol CAS No. 2445-53-6) not only aids in miscibility of the hyaluronic acid within the disclosed compositions but also synergistically interacts therewith to aid in topical and/or transdermal delivery to further improve skin hydration and to further stimulate fibroblasts within the dermis to synthesize collagen, resulting in firmer skin appear. The silanol and/or organolsilanol (e.g., CAS No.: 4253-34-3 or methylsilanetriol CAS No. 2445-53-6) is preferably included with the composition at approximately 0.5 wt % to 6 wt % of the overall composition and at a ratio ranging from approximately 3:1 to 1:1 (and more preferably from 2:1 to 1:1) of hyaluronic acid to the silanol and/or organolsilanol (e.g., CAS No.: 4253-34-3 or methylsilanetriol CAS 2445-53-6). In certain aspects, the low molecular weight hyaluronic acid (150 kDA to 600 kDA) disclosed herein may be crosslinked/bonded (e.g., covalently bonded) to the silanol and/or organolsilanol (e.g., CAS No.: 4253-34-3 or methylsilanetriol CAS 2445-53-6), which may further facilitate topical and transdermal absorption thereof. In certain aspects, the admixture of hyaluronic acid with silanol and/or organolsilanol (e.g., CAS No.: 4253-34-3 or methylsilanetriol CAS 2445-53-6) is Epidermosil manufactured by Exsymol S.A.M.

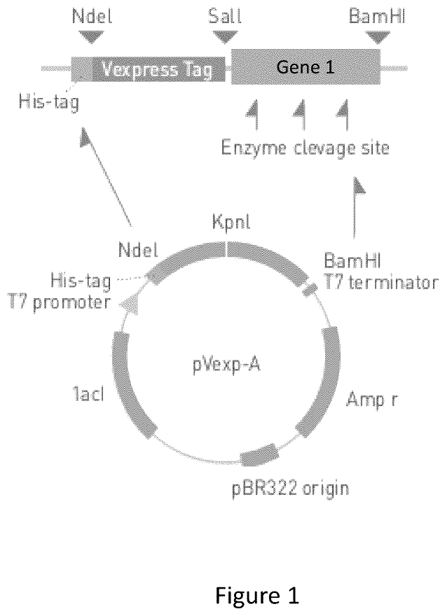

In certain aspects, each formulation and/or composition disclosed herein includes at least 5 but no more than 12 recombinant human growth factors. It should be noted, and further in view of FIG. 1, that recombinant human growth factors refers to transgenically produced and isolated polypeptides, which remain bioactive and bioavailable when included within the compositions disclosed herein. FIG. 1 specifically depicts a plasmid and/or vector in which the desired nucleotide sequence (indicated as “Gene 1”) may be ligated therein. Bacteria (e.g., E. coli) may be subsequently transformed with the plasmid and/or vector having the desired nucleotide sequence and may be subsequently expressed as a protein/polypeptide, which then may be subsequently isolated via techniques known in the art (e.g., tagging and affinity chromatography techniques). As previously alluded to above and when compared to formulations having growth factors derived from cultured media/medium, the recombinant human growth factors disclosed herein are less likely and/or will not induce an immunogenic response within a user because contaminants and/or immunogenic epitopes have been minimized and/or eliminated during the isolation thereof.

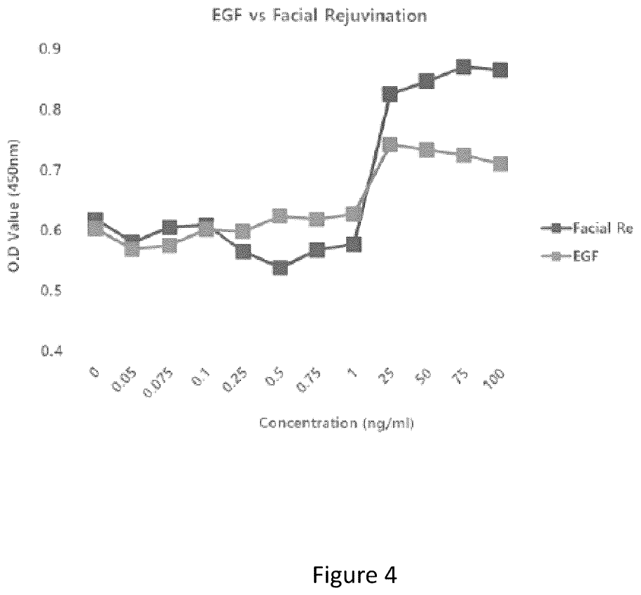

As alluded to above and as further shown in FIG. 4, multiple human recombinant growth factors when comparted to a single human recombinant growth factors exhibit a marked increase of cell proliferation further demonstrating that multiple human recombinant growth factors may synergistically interact to induce cell growth, proliferation, and repair, which may further lead to the desired results of repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin as disclosed herein. In this aspect, the recombinant human growth factors include at least 5 but no more than 12 of the following (also shown below in Table 1): sh-Polypeptide-1 (SEQ ID No. 2), sh-Polypeptide-11 (SEQ ID No. 3), sh-Polypeptide-31 (SEQ ID No. 4), sh-Oligopeptide-2 (SEQ ID No. 6), sh-Polypeptide-10 (SEQ ID No. 7), sh-Polypeptide-5 (SEQ ID No. 8), sh-Polypeptide-8 (SEQ ID No. 9), sh-Polypeptide-3 (SEQ ID No. 10), sh-Polypeptide-62 (SEQ ID No. 1), Acetyl Octapeptide-17 Amide (SEQ ID No. 5), sh-oligopeptide-1 (SEQ ID No. 11), sh-Polypeptide-4 (SEQ ID No. 12), and Acetyl sh-Oligopeptide-77 Amide (SEQ ID No. 13). A combination of these growth factors is used to stimulate endogenous proliferative and reparative molecular pathways. For example, if increase skin repair is desired, certain growth factors may be strategically selected from those listed in Table 1 to further activate endogenous mismatch repair pathways and/or to upregulate endogenous mismatch repair enzymes (e.g., MSH2, MSH3, MSH6, MLH1, MLH3, PMS1, PMS2, etc.) and/or base excision repair enzymes to stimulate DNA repair. The recombinant human growth factors disclosed herein were obtained from PnP Biopharm (Seoul, Korea). In certain aspects, the recombinant human growth factors of the composition are when sh-Polypeptide-1 is SEQ ID NO 2, sh-Polypeptide-11 is SEQ ID NO 3, sh-Polypeptide-31 is SEQ ID NO 4, shOligopeptide-2 is SEQ ID NO 6, the sh-Polypeptide-10 is SEQ ID NO 7, sh-Polypeptide-5 is SEQ ID NO 8, sh-Polypeptide-8 is SEQ ID NO 9, sh-Polypeptide-3 is SEQ ID NO 10, sh-Polypeptide-62 is SEQ ID NO 1, Acetyl Octapeptide-17 Amide is SEQ ID NO 5, sh-oligopeptide-1 is SEQ ID NO 11, sh-Polypeptide-4 is SEQ ID NO 12, and Acetyl sh-Oligopeptide-77 Amide is SEQ ID NO 13, with each growth factor being present at a concentration ranging from 0.001 wt % to 1 wt % of the overall composition and more preferably from 0.009 wt % to 1 wt %. In other aspects, the recombinant human growth factors of the composition are when sh-Polypeptide-1 comprises a sequence at least 90%, 95%, or 98% identical to SEQ ID NO 2, the sh-Polypeptide-11 comprises a sequence at least 90%, 95%, or 98% identical to SEQ ID NO 3, the sh-Polypeptide-31 comprises a sequence at least 90%, 95%, or 98% identical to SEQ ID NO 4, the shOligopeptide-2 comprises a sequence at least 90%, 95%, or 98% identical to SEQ ID NO 6, the sh-Polypeptide-10 comprises a sequence at least 90%, 95%, or 98% identical to SEQ ID NO 7, the sh-Polypeptide-5 comprises a sequence at least 90%, 95%, or 98% identical to SEQ ID NO 8, the sh-Polypeptide-8 comprises a sequence at least 90%, 95%, or 98% identical to SEQ ID NO 9, the sh-Polypeptide-3 comprises a sequence at least 90%, 95%, or 98% identical to SEQ ID NO 10, the sh-Polypeptide-62 comprises a sequence at least 90%, 95%, or 98% identical to SEQ ID NO 1, Acetyl Octapeptide-17 Amide comprises a sequence at least 90%, 95%, or 98% identical to SEQ ID NO 5, the sh-oligopeptide-1 comprises a sequence at least 90%, 95%, or 98% identical to SEQ ID NO 11, the sh-Polypeptide-4 comprises a sequence at least 90%, 95%, or 98% identical to SEQ ID NO 12, and the Acetyl sh-Oligopeptide-77 Amide comprises a sequence at least 90%, 95%, or 98% identical to SEQ ID NO 13, with each growth factor being present at a concentration ranging from 0.001 wt % to 1 wt % of the overall composition and more preferably from 0.009 wt % to 1 wt %.

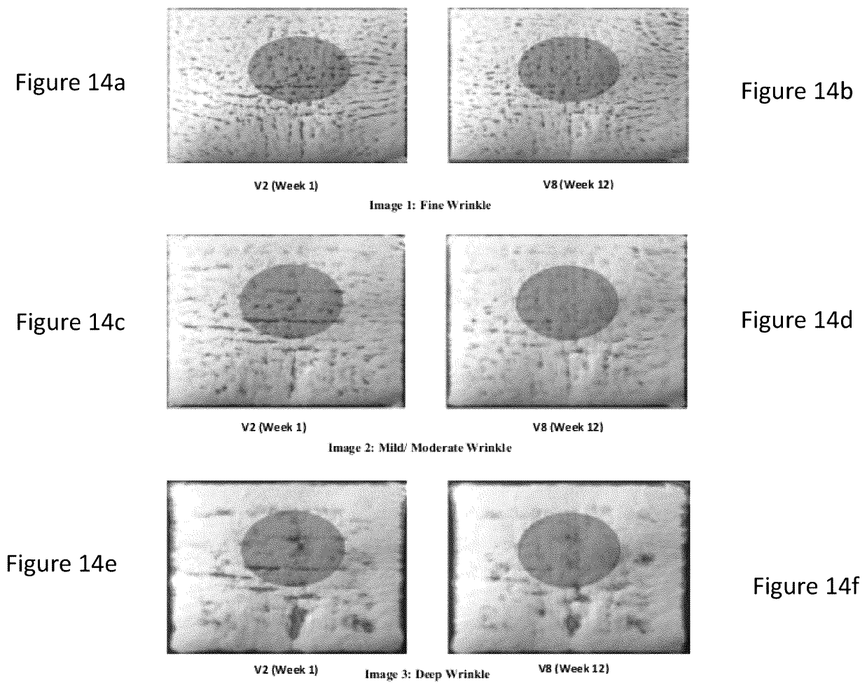

It should be further noted and to achieve, for example, the desired proliferative effects graphically depicted in FIG. 4 and/or as photographically shown in FIGS. 14a-14f, 16a-16f, 18a-18f, 20a-20f, 22a-22f, and 24a-24f, each growth factor, when present in the composition, is present at a concentration ranging from 0.001 wt % to 1 wt % of the overall composition and more preferably from 0.009 wt % to 1 wt %.

TABLE 1

| INCI Name | | MW (KDa) | Description |
|---|---|---|---|
| sh-Polypeptide-62 | HGF (Hepatocyte | 75 | HGF promotes migration of endothelial |
| (SEQ ID No. 1) | Growth Factor) | | cells enhances keratinocyte migration and |
| | | | proliferation * and has strong Anti- |
| | | | Inflammatory effects** |
| sh-Polypeptide-1 | Basic Fibroblast | 23.1 | bFGF (basic fibroblast factor) growth |
| (SEQ ID No. 2) | Growth Factor, | | promotes fibroblast and epithelial cell |
| | FGF2 | | proliferation, angiogenesis |
| sh-Polypeptide-11 | aFGF (acidic | 15.9 | aFGF (acidic fibroblast growth factor) |
| (SEQ ID No. 3) | Fibroblast Growth | | promotes fibroblast and epithelial cell |
| | Factor) | | proliferation, angiogenesis |
| sh-Polypeptide-31 | IGF2 (Insulin-like | 7.5 | ******The growth hormone/insulin-like |
| (SEQ ID No. 4) | Growth Factor-2) | | growth factor 1 and 2 (GH/IGF1) |
| | | | signaling pathway, also referred to as the |
| | | | somatotropic axis, has been extensively |
| | | | implicated in the aging process |
| Acetyl | Mini EGF | 1.14 | Smaller molecule to increase the |
| Octapeptide-17 | (Epidermal | | penetration into the skin. EGF (epidermal |
| Amide | Growth Factor) | | growth factor) produces cellular |
| (SEQ ID No. 5) | | | proliferation, differentiation, and survival |
| sh-Oligopeptide-2 | IGF1 (Insulin-like | 7.6 | IGF-1 (insulin-like growth factor 1) is the |
| (SEQ ID No. 6) | Growth Factor-1) | | major mediator of growth hormone |
| | | | stimulated somatic growth, and mediator |
| | | | of GH-independent anabolic responses |
| sh-Polypeptide-10 | FGF-10 | 23.1 | *****FGF has a relevant role in anti- |
| (SEQ ID No. 7) | (Fibroblast | | aging therapy because it is related to |
| | Growth Factor-10 | | collagen and elastin synthesis activation |
| | | | responsible for skin resistance and |
| | | | elasticity, characteristics that are |
| | | | diminished with skin aging |
| sh-Polypeptide-5 | TGF-β3 | 12.9 | TGF-beta 3 (transforming growth factor |
| (SEQ ID No. 8) | (Transforming | | beta 3) regulates epidermal and dermal |
| | Growth Factor-β3) | | cells in healing skin, modulates |
| | | | inflammation and reduces scar formation |
| sh-Polypeptide-8 | PDGF (Platelet- | 14.3 | PDGF has been proven to be anti- |
| (SEQ ID No. 9) | Derived Growth | | inflammatory and is a required element in |
| | Factor | | cellular division for fibroblasts |
| sh-Polypetpide-3 | KGF | 16.2 | KGF enhances the effect of EGF**** |
| (SEQ ID No. 10) | (Keratinocyte | | and ensures the proliferation of |
| | Growth Factor) | | keratinocytes |
| Sh- oligopeptide-1 | EGF-1 | 6.3 | EGF (epidermal growth factor) produces |
| (SEQ ID No. 11) | (Epidermal | | cellular proliferation, differentiation, and |
| | Growth Factor 1) | | survival |
| sh-Polypeptide-4 | Stem Cell Factor | 18.5 | Androgens appear to reduce alopecia hair |
| (SEQ ID No. 12) | | | colour by inhibiting dermal papilla SCF |
| | | | production, impeding bulbar melanocyte |
| | | | pigmentation. SCF can therefore increase |
| | | | the potential for reversing alopecia and |
| | | | hair colouring¹ |
| Acetyl sh- | Mini Noggin | 1.3 | Noggin² is a bone morphogenetic protein |
| Oligopeptide-77 | | | antagonist and is essential for hair follicle |
| Amide | | | growth phase induction. |
| (SEQ ID No. 13) | | | |

¹Randall VA¹, Jenner TJ; Stem cell factor/c-Kit signalling in normal and androgenetic alopecia hair follicles; Endocrinol. 2008 Apr; 197(1): 11-23.
²9Botchkarev VA¹, Botchkareva NV; Noggin is required for induction of the hair follicle growth phase in postnatal skin. FASEB J. 2001 Oct; 15(12): 2205-14.

As protein molecular weight increases, transdermal absorption and/or skin penetration is hindered and/or reduced. For example, transdermal absorption and/or skin penetration of growth factors tends to be effected when molecular weights exceed 500 Da (0.5 KDa). Furthermore, because of the delicate nature (disulfide bonding, protein folding, etc.) of recombinant human growth factors disclosed herein, these recombinant human growth factors may be susceptible to denaturation and/or decreased bioavailability and bioactivity if improperly stored. To increase transdermal absorption and/or skin penetration of the growth factors disclosed herein while further decreasing susceptibility to denaturation (and/or to maintain optimal bioavailability and bioactivity), the recombinant human growth factors disclosed herein are preferably nanoencapsulated with a nanoencapsulating agent that encapsulates the recombinant human growth factors therein. The nanoencapsulating agent is preferably present in the composition at an effective amount to deliver the recombinant human growth factors to cells, keratinous materials, and/or the extracellular matrix in a human.

In certain aspects, the nanoencapuslating agent preferably includes a mixture of a C1-C6 alkylene glycol (e.g., diols or polyols) suitable for topical and/or transdermal use in combination with lecithin. In this mixture, each component is present at a ratio ranging from 4:1 to 2.3:1 of C1-C6 alkylene glycol to lecithin (CAS No. 8002-43-5). The C1-C6 alkylene glycol suitable for topical and/or transdermal use is present at a concentration of 70 wt % to 80 wt % of the overall concentration of the nanoencapsulating agent and lecithin is present at a concentration of 20 to 30 wt % of the overall concentration of the nanoencapsulating agent. In certain aspects, the C1-C6 alkylene glycol is ethylene glycol, propanediol (propylene glycol, propane 1,2 diol) (CAS No. 504-63-2) or a combination thereof, and in preferred aspects, the C1-C6 alkylene glycol is propanediol (propylene glycol, propane 1,2 diol) present at a concentration of 70 wt % to 80 wt % of the overall concentration of the nanoencapsulating agent. It should be further noted that the lecithin in the nanoencapsulating agent is a soybean (G. max) extract (or alternatively a plurality of isolates that are combined with one another) in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanoloamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent. The nanoencapsulating agent may include additives including, for example, tocopherol at a concentration ranging from 0.15 wt % to 0.35 wt % of the overall concentration of nanoencapsulating agent and sunflower (Helianthus annuus) seed oil at a concentration ranging from 0.09 wt % to 0.25 wt % of the overall concentration of nanoencapsulating agent. In certain aspects, the nanoencapsulating agent may be Pro-Lip™Neo from Lucas Meyer Cosmetics S.A.S.

In certain aspects, all compositions/formulations disclose herein are preferably pH balanced having a pH ranging from 5.5 to 7.5 to reduce and completely avoid skin irritation attributed to application thereon. To obtain this pH balance, salt solutions including, for example, a phosphate buffered saline such as KH₂PO₄/K₂HPO₄ saline buffer may be used.

Regenerative Composition/Formulation

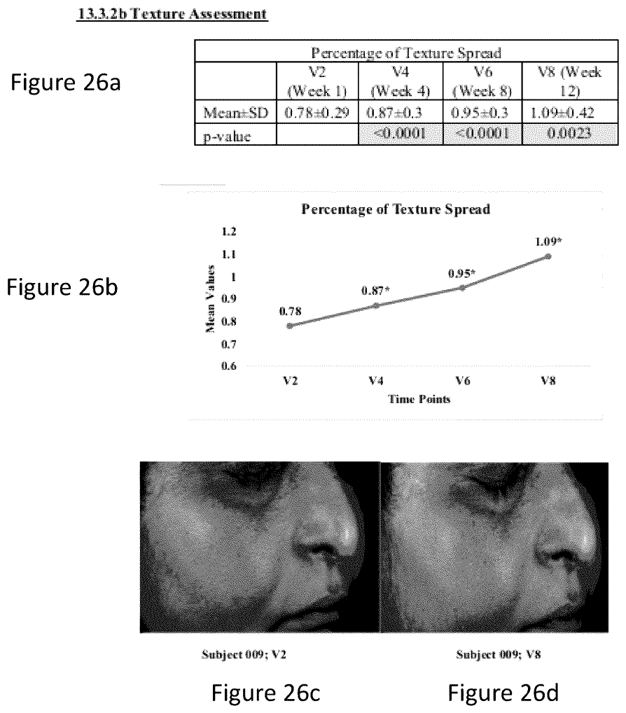

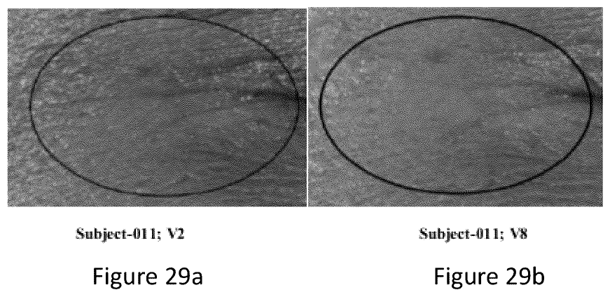

In certain aspects, an objective is to provide an aqueous solution for topical use (and transdermal penetration) that stimulates and improves keratinocyte proliferation, epidermis regeneration, and thickness to provide a more youthful appearance that resurfaces skin, increases skin tightness, decreases pore size, improves skin texture, and reduces vertical wrinkles, which is shown, for example, in FIGS. 26c, 26d, 27c, 27d, 29a, and 29b.

In this aspect, the aqueous solution for topical use includes water as a solvent at the concentrations discussed above (ranging from 55 wt % to 85 wt %) regarding the general composition/formulation, but more preferably includes from 70 wt % to 85 wt % water of the overall concentration of this composition/formulation, and most preferably includes from 75 wt % to 82.5 wt % water of the overall concentration of this composition/formulation.

In certain optional embodiments, this composition may or may not include hyaluronic acid (CAS No. 9067-32-7) and silanol and/or organosilanol (CAS No.: 4253-34-3) is methylsilanetriol (CAS No. 2445-53-6), at substantially the same concentrations as those previously discussed above. However, in certain embodiments regarding the regenerative compositions/formulations and as shown in Table 2 below, hyaluronic acid and the organosilanol is preferably excluded.

This composition further includes preferably at least eight of the previously mentioned recombinant human growth factors and up to twelve of the previously mentioned recombinant human growth factors with each growth factor, when present in the composition, is present at a concentration ranging from 0.001 wt % to 1 wt % of the overall composition and more preferably from 0.009 wt % to 1 wt %. The growth factors are selected to specifically achieve the above mentioned, desired effects (i.e., stimulate and improve keratinocyte proliferation, epidermis regeneration, and thickness to provide a more youthful appearance that potentially resurfaces skin, skin tightness, pore size, skin texture, and potentially reduces vertical wrinkles). In certain aspects, this composition/formulation, preferably includes each of the following recombinant human growth factors: sh-Polypeptide-1 (SEQ ID No. 2), sh-Polypeptide-11 (SEQ ID No. 3), sh-Polypeptide-31 (SEQ ID No. 4), sh-Oligopeptide-2 (SEQ ID No. 6), sh-Polypeptide-10 (SEQ ID No. 7), sh-Polypeptide-5 (SEQ ID No. 8), sh-Polypeptide-8 (SEQ ID No. 9), sh-Polypeptide-3 (SEQ ID No. 10), sh-Polypeptide-62 (SEQ ID No. 1), and Acetyl Octapeptide-17 Amide (SEQ ID No. 5), with each growth factor being present at a concentration ranging from 0.001 wt % to 1 wt % of the overall composition and more preferably from 0.009 wt % to 1 wt %. In certain aspects, this composition includes at least ten of the above mentioned recombinant human growth factors. In certain aspects, the recombinant human growth factors of the composition are when sh-Polypeptide-1 is SEQ ID NO 2, sh-Polypeptide-11 is SEQ ID NO 3, sh-Polypeptide-31 is SEQ ID NO 4, shOligopeptide-2 is SEQ ID NO 6, the sh-Polypeptide-10 is SEQ ID NO 7, sh-Polypeptide-5 is SEQ ID NO 8, sh-Polypeptide-8 is SEQ ID NO 9, sh-Polypeptide-3 is SEQ ID NO 10, sh-Polypeptide-62 is SEQ ID NO 1, Acetyl Octapeptide-17 Amide is SEQ ID NO 5, sh-oligopeptide-1 is SEQ ID NO 11, sh-Polypeptide-4 is SEQ ID NO 12, and Acetyl sh-Oligopeptide-77 Amide is SEQ ID NO 13, with each growth factor being present at a concentration ranging from 0.001 wt % to 1 wt % of the overall composition and more preferably from 0.009 wt % to 1 wt %. In other aspects, the recombinant human growth factors of the composition are when sh-Polypeptide-1 comprises a sequence at least 90%, 95%, or 98% identical to SEQ ID NO 2, the sh-Polypeptide-11 comprises a sequence at least 90%, 95%, or 98% identical to SEQ ID NO 3, the sh-Polypeptide-31 comprises a sequence at least 90%, 95%, or 98% identical to SEQ ID NO 4, the shOligopeptide-2 comprises a sequence at least 90%, 95%, or 98% identical to SEQ ID NO 6, the sh-Polypeptide-10 comprises a sequence at least 90%, 95%, or 98% identical to SEQ ID NO 7, the sh-Polypeptide-5 comprises a sequence at least 90%, 95%, or 98% identical to SEQ ID NO 8, the sh-Polypeptide-8 comprises a sequence at least 90%, 95%, or 98% identical to SEQ ID NO 9, the sh-Polypeptide-3 comprises a sequence at least 90%, 95%, or 98% identical to SEQ ID NO 10, the sh-Polypeptide-62 comprises a sequence at least 90%, 95%, or 98% identical to SEQ ID NO 1, Acetyl Octapeptide-17 Amide comprises a sequence at least 90%, 95%, or 98% identical to SEQ ID NO 5, the sh-oligopeptide-1 comprises a sequence at least 90%, 95%, or 98% identical to SEQ ID NO 11, the sh-Polypeptide-4 comprises a sequence at least 90%, 95%, or 98% identical to SEQ ID NO 12, and the Acetyl sh-Oligopeptide-77 Amide comprises a sequence at least 90%, 95%, or 98% identical to SEQ ID NO 13, with each growth factor being present at a concentration ranging from 0.001 wt % to 1 wt % of the overall composition and more preferably from 0.009 wt % to 1 wt %.

The recombinant human growth factors mentioned herein for the regenerative composition/formulation are also nanoencapsulated with the same nanoencapsulating agent(s) mentioned above at substantially the same concentrations and for substantially the same reasons as those mentioned above regarding the general composition/formulation.

In certain aspects, the aqueous solution for topical use further includes a Swertia chirata extract present at an effective amount for stimulating endogenous keratinocyte growth factor production to induce keratinocyte proliferation and epidermis regeneration to increase skin volume and/or reduce cutaneous signs of aging. In certain aspects, this Swertia chirata extract may be present at a concentration ranging from 1 wt % to 8 wt % of the overall composition, and more preferably from 2.5 wt % to 5 wt % of the overall composition. In particular the Swertia chirata extract when present at the concentrations mentioned above and coupled with the recombinant human growth factors acting through a biomimetic pathway to stimulate keratinocyte growth factor (KGF) production from adipose-derived stem cells (ADSC), which advantageously improves keratinocyte proliferation and epidermis regeneration and thickness.

The aqueous solution further includes skin protecting agents that include an admixture of glycerin, water, dextran, and caproyl tetrapeptide-3 at a concentration ranging from 1 wt % to 5 wt % of the overall composition, and more preferably from 2.5 wt % to 4 wt % of the overall composition. The skin protecting agent disclosed immediately above, when included within the aqueous solution for topical use, advantageously stimulates the production of key components at the dermo-epidermal junction (collagen VII, laminin-5, and fibronectin).

The aqueous solution further includes niacinamide (vitamin B₃)(CAS No. 98-92-0) at a concentration ranging from 0.1 wt % to 5 wt % of the overall composition, and more preferably 1.5 wt % to 3.5 wt % of the overall composition. When present at these concentrations, niacinamide advantageously minimizes enlarged pores, tighten lax pores, improves uneven skin tone, soften fine lines and wrinkles, diminishes skin dullness, and strengthens the skin surface.

The remainder of the aqueous solution for topical use further includes additional conditioners, emollients, and viscosity controlling agents. For example, in certain aspects, the emollients may include a mixture of polyacrylate-13, polyisobutylene, and polysorbate 20 at a concentration ranging from 1 to 5 wt % of the overall composition, and more preferably from 2 wt % to 3 wt % of the overall composition. The viscosity controlling agent is carnosine at a concentration ranging from 0.1 wt % to 1 wt % of the overall composition, and more preferably from 0.1 wt % to 0.3 wt % of the overall composition. The additional conditioners may further include a mixture of disodium EDTA, glycerin, and jojoba leaf extract at a concentration ranging from 0.02 wt % to 2 wt % of the overall composition, and more preferably from 0.05 wt % to 1 wt % of the overall composition.

This composition may be used alone or in conjunction with the other compositions/formulations disclosed herein as well as with microneedling procedures as disclosed below in the “Methods of Use” to improve the user's appearance. An exemplary regenerative composition/formulation is shown immediately below within Table 2.

TABLE 2
REGENERATIVE SOLUTION

| No | INGREDIENTS | Function | Range |
|---|---|---|---|
| 1 | Water (Aqua) | Solvent | 60-80% |
| 2 | Maltodextrin, | Various | 1-8% |
| | Swertia Chirata | | |
| | Extract | | |
| 3 | Glycerin (and) | Skin protecting | 1-5% |
| | Water (and) | | |
| | Dextran (and) | | |
| | Caproyl | | |
| | Tetrapeptide-3 | | |
| 4 | Niacinamide | Skin | 0.1-5% |
| | | conditioning | |
| 5 | Cetyl | Smoothing | 2-4% |
| | Ethylhexanoate | | |
| 6 | Polyacrylate-13 | Emollient | 1-5% |
| | (and) | | |
| | Polyisobutylene | | |
| | (and) Polysorbate | | |
| | 20 | | |

TABLE 2
REGENERATIVE SOLUTION

| No | INGREDIENTS | Function | Range |
|---|---|---|---|
| 7 | Dimethyl | Binding | 1-5% |
| | Isosorbide | | |
| 8 | Xylityl | Anti-microbial | 1-5% |
| | Sesquicaprilate | | |
| | (and) Caprylyl | | |
| | Glycol | | |
| 9 | Carnosine | Viscosity | 0.1-1% |
| | | controlling | |
| 10 | Water, Pentylene | Surfactant, | 0.001%-1% |
| | Glycol, 1,2 | Growth | |
| | Hexanediol, | Factors | |
| | Sodium | | |
| | Phosphate, | | |
| | Lecithin, Sh- | | |
| | Polypeptide-1, sh- | | |
| | Polypeptide-11, | | |
| | sh-Polypeptide- | | |
| | 31, sh- | | |
| | Oligopeptide-2, | | |
| | sh-Polypeptide-10, | | |
| | sh-Polypeptide-5, | | |
| | sh-Polypeptide-8, | | |
| | sh-Polypeptide-3, | | |
| | sh-Polypeptide-62, | | |
| | Acetyl | | |
| | Octapeptide-17 | | |
| | Amide | | |
| 11 | Disodium EDTA | Conditioner | 0.01-1% |
| 12 | Glycerin (and) | Conditioner | 0.01-1% |
| | Water (and) | | |
| | Simmondsia | | |
| | Chinensis (Jojoba | | |
| | Leaf Extract) | | |

Reparative Composition/Formulations

In certain aspects, the reparative compositions/formulations are oil in water emulsion for topical use and transdermal absorption disclosed herein are specifically tailored for treating skin damage, wrinkles, etc. resulting from exposure (or over exposure) to, for example, UV. To achieve this result, these compositions include the specific combination of human recombinant growth factors discussed below as well as a specific combination of enzymes (e.g., photolyases, plant-derived roxisomes, and bacterial endonucleases) as well as various anti-inflammatory agents and anti-oxidants that further activate and up-regulate various DNA repair pathways (e.g., mismatch repair pathways to remove mismatch DNA nucleotides, base-excision repair pathways to remove damaged DNA/improperly paired nucleotides (e.g., oxidized guanine resulting in improper Hoogsteen base pairing with adenine, alkylated nucleotides, and/or deaminated nucleotides) often associated with UV/DNA damage, and/or DNA damage resulting from reactive oxygen species (ROS)) to more quickly repair DNA damage (which may be associated with photoaging) and to further mitigate, reduce, and/or improve skin appearance.

In this aspect, the reparative compositions/formulations includes water as a solvent at the concentrations discussed above (ranging from 55 wt % to 85 wt %) regarding the general composition/formulation, but more preferably includes from 70 wt % to 85 wt % water of the overall concentration of this composition/formulation, and most preferably includes from 75 wt % to 82.5 wt % water of the overall concentration of this composition/formulation.

This composition includes hyaluronic acid (CAS No. 9067-32-7) and silanol and/or organosilanol (CAS No.: 4253-34-3) is methylsilanetriol (CAS No. 2445-53-6), at substantially the same concentrations as those discussed above and as further shown in Table 3 below. In certain aspects, the hyaluronic acid is present at 1 to 3 wt % of the overall composition and the organosilanol (e.g., mehylsilanetriol) is present at 1 to 3 wt % of the overall composition.

This composition further includes preferably at least eight of the previously mentioned recombinant human growth factors and up to twelve of the previously mentioned recombinant human growth factors with each growth factor, when present in the composition, is present at a concentration ranging from 0.001 wt % to 1 wt % of the overall composition and more preferably from 0.009 wt % to 1 wt %. The growth factors are selected to specifically achieve the above mentioned, desired effects (i.e., stimulate and improve keratinocyte proliferation, epidermis regeneration, and thickness to provide a more youthful appearance that potentially resurfaces skin, skin tightness, pore size, skin texture, and potentially reduces vertical wrinkles). In certain aspects, this composition/formulation, preferably includes each of the following recombinant human growth factors: sh-Polypeptide-1 (SEQ ID No. 2), sh-Polypeptide-11 (SEQ ID No. 3), sh-Polypeptide-31 (SEQ ID No. 4), sh-Oligopeptide-2 (SEQ ID No. 6), sh-Polypeptide-10 (SEQ ID No. 7), sh-Polypeptide-5 (SEQ ID No. 8), sh-Polypeptide-8 (SEQ ID No. 9), sh-Polypeptide-3 (SEQ ID No. 10), sh-Polypeptide-62 (SEQ ID No. 1), and Acetyl Octapeptide-17 Amide (SEQ ID No. 5), with each growth factor being present at a concentration ranging from 0.001 wt % to 1 wt % of the overall composition and more preferably from 0.009 wt % to 1 wt %. In certain aspects, this composition includes at least ten of the above mentioned recombinant human growth factors. In certain aspects, the recombinant human growth factors of the composition are when sh-Polypeptide-1 is SEQ ID NO 2, sh-Polypeptide-11 is SEQ ID NO 3, sh-Polypeptide-31 is SEQ ID NO 4, shOligopeptide-2 is SEQ ID NO 6, the sh-Polypeptide-10 is SEQ ID NO 7, sh-Polypeptide-5 is SEQ ID NO 8, sh-Polypeptide-8 is SEQ ID NO 9, sh-Polypeptide-3 is SEQ ID NO 10, sh-Polypeptide-62 is SEQ ID NO 1, Acetyl Octapeptide-17 Amide is SEQ ID NO 5, sh-oligopeptide-1 is SEQ ID NO 11, sh-Polypeptide-4 is SEQ ID NO 12, and Acetyl sh-Oligopeptide-77 Amide is SEQ ID NO 13, with each growth factor being present at a concentration ranging from 0.001 wt % to 1 wt % of the overall composition and more preferably from 0.009 wt % to 1 wt %. In other aspects, the recombinant human growth factors of the composition are when sh-Polypeptide-1 comprises a sequence at least 90%, 95%, or 98% identical to SEQ ID NO 2, the sh-Polypeptide-11 comprises a sequence at least 90%, 95%, or 98% identical to SEQ ID NO 3, the sh-Polypeptide-31 comprises a sequence at least 90%, 95%, or 98% identical to SEQ ID NO 4, the shOligopeptide-2 comprises a sequence at least 90%, 95%, or 98% identical to SEQ ID NO 6, the sh-Polypeptide-10 comprises a sequence at least 90%, 95%, or 98% identical to SEQ ID NO 7, the sh-Polypeptide-5 comprises a sequence at least 90%, 95%, or 98% identical to SEQ ID NO 8, the sh-Polypeptide-8 comprises a sequence at least 90%, 95%, or 98% identical to SEQ ID NO 9, the sh-Polypeptide-3 comprises a sequence at least 90%, 95%, or 98% identical to SEQ ID NO 10, the sh-Polypeptide-62 comprises a sequence at least 90%, 95%, or 98% identical to SEQ ID NO 1, Acetyl Octapeptide-17 Amide comprises a sequence at least 90%, 95%, or 98% identical to SEQ ID NO 5, the sh-oligopeptide-1 comprises a sequence at least 90%, 95%, or 98% identical to SEQ ID NO 11, the sh-Polypeptide-4 comprises a sequence at least 90%, 95%, or 98% identical to SEQ ID NO 12, and the Acetyl sh-Oligopeptide-77 Amide comprises a sequence at least 90%, 95%, or 98% identical to SEQ ID NO 13, with each growth factor being present at a concentration ranging from 0.001 wt % to 1 wt % of the overall composition and more preferably from 0.009 wt % to 1 wt %.

The recombinant human growth factors mentioned herein for the regenerative composition/formulation are also nanoencapsulated with the same nanoencapsulating agent(s) mentioned above at the same concentrations and for substantially the same reasons as those mentioned above regarding the general composition/formulation.

In addition and to further promote DNA repair (e.g., via base excision repair and/or mismatch repair pathways) and reduction and repair of UV associated photoaging, this composition further include a combination of liposomally encapuslated bacterial derived photolyase(s), plant-derived roxisome(s), and bacterial derived endonuclease(s) that are present in an effective amount prevent and/or reduce photoaging associated with ultraviolet (UV) exposure by enhancing endogenous DNA repair. The combination of liposomally encapuslated bacterial derived photolyase(s), plant-derived roxisome(s), and bacterial derived endonucleases are present at a concentration ranging from 0.3 wt % to 10 wt % of the overall composition. In certain aspects, the bacterial derived photolyases (also referred to as “photosomes”) are present at a concentration ranging from 0.1 wt % to 3.5 wt % of the overall composition, and preferably are bacterial derived photolyase is a cyanobacteria photolyase in which the cyanobacteria photolyase is an Anacystis nidulans photolyase. The photolyases are generally referred to and may be obtained as “photosomes” from Barnet Products Corporation and includes a mixture of plankton extract (CAS No. 37290-70-3) and lecithin (8002-43-5) that encapsulates the plankton extract therein. Within this composition, plant derived roxisome(s) are present at a concentration ranging from 0.1 wt % to 3.5 wt % of the overall composition in order to combat DNA damage resulting from endogenous reactive oxygen species (ROS) that oxidize guanine into 8-oxo-guanine. These plant derived roxisome(s) are preferably 8-oxo-guanine glycosylase from A. thaliana and may be further obtained from Barnet Products Corporation generally as “Roxisomes” (including a mixture of A. thaliana extract (CAS No. 89958-13-4), lecithin (CAS No. 8002-43-5), and water (CAS No. 7732-18-5)). Bacterial derived endonucleases (also referred to as “Ultrasomes”) are present at a concentration ranging from 0.1 wt % to 3.5 wt % of the overall composition in which the bacterial derived endonuclease(s) are bacterial endonucleases from M. Luteus and may further reduce expression of tumor necrosis factor (TNF) and interleukin-1, 6, and 8 often associated with UV damage. These ultrasomes may also be obtained from Barnet Products. In certain aspects, the liposomal encapsulating agent that encapsulates each of the derived photolyase(s), plant-derived roxisome(s), and bacterial derived endonuclease(s) may be lecithin and may be substantially identical to the nanoencapsulating agent(s) disclosed above. Furthermore, in certain aspects, the photolyase(s), plant-derived roxisome(s), and bacterial derived endonuclease(s) may be encapsulated within the same micelles the recombinant human growth factors within these compositions.

To further enhance this compositions reparative characteristics a plurality of anti-inflammatory agents and/or antioxidants at a concentration ranging from 1 wt % to 10 wt % of the overall composition is also included, which aid in reducing the deleterious effects of ROS and/or mitigate and/or reduce endogenous inflammatory pathways. For example, within this plurality of anti-inflammatory agents and/or antioxidants an Evodia rutaecarpa fruit extract (e.g., Evodiox®) is present at a concentration ranging from 0.1 wt % to 3 wt % of the overall composition (and more preferably 0.5 wt % to 3 w %); this extract exhibits strong anti-inflammatory properties. In addition, thiotaine (INCI Name: Ergothioneine; CAS No. 497-30-3) is preferably included at a concentration ranging from 0.1 to 3 wt % of the overall composition (and more preferably 0.5 wt % to 3 wt %), which further mitigates and/or aids in repair of DNA damage associated with ROS, especially ROS that oxidize guanine into 8-oxo-guanine leading to Hoogsteen base pairing during, for example, DNA replication. Retinol is also included within the plurality of anti-inflammatory agents and/or antioxidants at a concentration ranging from 1 wt % to 3 wt % of the overall concentrations.

Additional additives, emollients, preservatives, and conditioners may also be included and comprise the remainder of the reparative composition/formulation. For example, in certain aspects, this composition includes emollient and an emulsifier present at a concentration ranging from 6 wt % to 32 wt % of the overall composition

This composition may be used alone or in conjunction with the other compositions/formulations disclosed herein as well as with microneedling procedures as disclosed below in the “Methods of Use”. An exemplary reparative composition/formulation is shown immediately below within Table 3.

TABLE 3
REPARATIVE SOLUTION

| INGREDIENTS | Comment | Range |
|---|---|---|
| Water (aqua) | | 60-80% |
| Capric/Caprylic Triglyceride | Emollient | 2-10% |
| Candelilla/Jojoba/Rice Bran | EMULIUM ® KAPPA MB is a | 2-10% |
| Polyglyceryl-3 Esters (and) | natural, PEG-free, O/W sensory | |
| Glyceryl Stearate (and) | emulsifier. Designed to bring a | |
| Cetearyl Alcohol (and) | silicone feel to natural formulations, | |
| Sodium Stearoyl Lactylate | creams and lotions created with | |
| | EMULIUM ® KAPPA MB are soft, | |
| | rich, and luxurious. EMULIUM ® | |
| | KAPPA MB also creates active | |
| | textures by bring moisturization to the | |
| | skin. Certified natural by ECOCERT, | |
| | NSF and RSPO Mass Balance. | |
| BPS COMPLEX | BPS complex is an optimized blend of | 2-10% |
| | natural lipds. It imparts a luxurious | |
| | emolliency without greasiness. | |
| Silanetriol (and) Hyaluronic | | 1-5% |
| Acid | | |
| Cetearyl Alcohol | | 1-5% |
| Chondrus Crispus Extract | | 1-5% |
| Butylene Glycol | | 1-5% |
| Glycerin | | 1-5% |
| Dimethicone | | 1-5% |
| Plukenetia Volubilis Seed | PHYTO-CERAMIDYL OMEGA N is | 1-5% |
| Oil, Olea Europaea (Olive) | a complete protective treatment for | |
| Fruit Oil, Aqua (Water), | weak hair and dry, mature and | |
| Glycerin, Ceramide NP, | sensitive skin. Increases | |
| Polyglyceryl-.10 laureate, | Moistirusation of aged skin | |
| Citric acid, Tocopherol | | |
| Vitis Vinifera (Grape) Seed | | 0.1-3% |
| Oil | | |
| Butyrospermum Parkii (Shea) | | 0.1-3% |
| Butter | | |
| Phenoxyethanol (and) | Preservative | 0.1-3% |
| Glycerol (and) Caprylyl | | |
| Glycol (and) | | |
| Ethylhexylglycerin (and) | | |
| Hexylene Glycol | | |
| EVODIOX | Evodiox is a strong anti-inflammatory | 0.1-3% |
| | and is derived from the fruit of the | |
| | Chinese Evodia plant*. It has been | |
| | proven reduce inflammation by 40% | |
| ROXISOMES | Concerns over existing sunscreen | 0.1-3% |
| ULTRASOMES | filters have reinforced the need to | 0.1-3% |
| PHOTOSOMES | examine supplemental sun protection | 0.1-3% |
| | or repair of sun damage. Technology | |
| | to enhance DNA repair has been | |
| | available in skincare and sunscreen | |
| | products for several decades, but | |
| | skepticism and lack of familiarity with | |
| | the supporting data remain prevalent. | |
| | Here, we address six of the main | |
| | questions raised by medical | |
| | professionals regarding the efficacy of | |
| | DNA repair enzymes in sun | |
| | protection. These include the mode of | |
| | delivery and mechanism of action, the | |
| | effect on cellular responses and the | |
| | amelioration of pre-cancers, cancers | |
| | and photoaging. The conclusions are | |
| | that topical DNA repair enzymes do | |
| | enhance removal of DNA damage and | |
| | reduce the appearance of new actinic | |
| | keratoses as well as increase | |
| | regression of existing lesions. Support | |
| | for prevention of photoaging and skin | |
| | cancer is significant** | |
| THIOTAINE | A strong anti-oxidant. It shields | 0.1-3% |
| | against 8 oxxGuanine oxidative DNA | |
| | which is conventionally difficult to | |
| | treat | |
| Tetrahexyldecyl Ascorbate | | 0.01-1% |
| Magnesium Aluminum | | 0.01-1% |
| Silicate | | |
| Retinol | | 1-3% |
| Xanthan Gum | | 0.001-1% |
| Tocopheryl Acetate | | 0.001-1% |
| Simmondsia Chinensis | | 0.001-1% |
| (Jojoba) Seed Oil | | |
| Panthenol | | 0.001-1% |
| Water, Pentylene glycol, 1,2 | Recombinant Human Growth Factors | 0.001-1% |
| Hexanediol, Sodium | | |
| Phosphate, Lecithin, Sh- | | |
| Polypeptide-1, sh- | | |
| Polypeptide-11, sh- | | |
| Polypeptide-31, sh- | | |
| Oligopeptide-2, sh- | | |
| Polypeptide-10, sh- | | |
| Polypeptide-5, sh- | | |
| Polypeptide-8, sh- | | |
| Polypeptide-3, sh- | | |
| Polypeptide-62, Acetyl | | |
| Octapeptide-17 Amide | | |
| Tetrasodium EDTA | | 0.0001-1% |

*Ko HC1, Wang YH; Anti-inflammatory effects and mechanisms of the ethanol extract of Evodia rutaecarpa and its bioactive components on neutrophils and microglial cells. Eur J Pharmacol. 2007 Jan. 26; 555(2-3): 211-7. Epub 2006 Oct. 17.
**Daniel B Yarosh, Amanda Rosenthal, and Ronald Moy; Six critical questions for DNA repair enzymes in skincare products: a review in dialog; Clin Cosmet Investig Dermatol. 2019; 12: 617-624.

Hair/Scalp Composition/Formulation

Also disclosed is an aqueous solution for topical use on a human scalp comprising at least 10 recombinant human growth factors (and up to 12 recombinant growth factors), and a nanoencapsulating agent that encapsulates the recombinant human growth factors and present in an effective amount to deliver the recombinant human growth factors to the human scalp to rejuvenate scalp appearance as well as to improve and/or restore hair growth.

In this aspect, the hair/scalp compositions/formulations include water as a solvent at the concentrations discussed above (ranging from 90 wt % to 98 wt %) regarding the general composition/formulation, but more preferably includes from 95 wt % to 97.5 wt % water of the overall concentration of this composition/formulation.

In this composition, at least 10 recombinant human growth factors are present and are selected from the following: sh-Polypeptide-1 (SEQ ID No. 2), sh-Polypeptide-11 (SEQ ID No. 3), sh-Polypeptide-31 (SEQ ID No. 4), sh-Oligopeptide-2 (SEQ ID No. 6), sh-Polypeptide-10 (SEQ ID No. 7), sh-Polypeptide-5 (SEQ ID No. 8), sh-Polypeptide-8 (SEQ ID No. 9), sh-Polypeptide-3 (SEQ ID No. 10), sh-Polypeptide-62 (SEQ ID No. 1), Acetyl Octapeptide-17 Amide (SEQ ID No. 5), sh-oligopeptide-1 (SEQ ID No. 11), sh-Polypeptide-4 (SEQ ID No. 12), and Acetyl sh-Oligopeptide-77 Amide (SEQ ID No. 13). When present in the composition, each growth is included at a concentration ranging from 0.001 wt % to 1 wt % of the overall composition and more preferably from 0.009 wt % to 1 wt %.. In certain aspects, the recombinant human growth factors of the composition are when sh-Polypeptide-1 is SEQ ID NO 2, sh-Polypeptide-11 is SEQ ID NO 3, sh-Polypeptide-31 is SEQ ID NO 4, shOligopeptide-2 is SEQ ID NO 6, the sh-Polypeptide-10 is SEQ ID NO 7, sh-Polypeptide-5 is SEQ ID NO 8, sh-Polypeptide-8 is SEQ ID NO 9, sh-Polypeptide-3 is SEQ ID NO 10, sh-Polypeptide-62 is SEQ ID NO 1, Acetyl Octapeptide-17 Amide is SEQ ID NO 5, sh-oligopeptide-1 is SEQ ID NO 11, sh-Polypeptide-4 is SEQ ID NO 12, and Acetyl sh-Oligopeptide-77 Amide is SEQ ID NO 13, with each growth factor being present at a concentration ranging from 0.001 wt % to 1 wt % of the overall composition and more preferably from 0.009 wt % to 1 wt %. In other aspects, the recombinant human growth factors of the composition are when sh-Polypeptide-1 comprises a sequence at least 90%, 95%, or 98% identical to SEQ ID NO 2, the sh-Polypeptide-11 comprises a sequence at least 90%, 95%, or 98% identical to SEQ ID NO 3, the sh-Polypeptide-31 comprises a sequence at least 90%, 95%, or 98% identical to SEQ ID NO 4, the shOligopeptide-2 comprises a sequence at least 90%, 95%, or 98% identical to SEQ ID NO 6, the sh-Polypeptide-10 comprises a sequence at least 90%, 95%, or 98% identical to SEQ ID NO 7, the sh-Polypeptide-5 comprises a sequence at least 90%, 95%, or 98% identical to SEQ ID NO 8, the sh-Polypeptide-8 comprises a sequence at least 90%, 95%, or 98% identical to SEQ ID NO 9, the sh-Polypeptide-3 comprises a sequence at least 90%, 95%, or 98% identical to SEQ ID NO 10, the sh-Polypeptide-62 comprises a sequence at least 90%, 95%, or 98% identical to SEQ ID NO 1, Acetyl Octapeptide-17 Amide comprises a sequence at least 90%, 95%, or 98% identical to SEQ ID NO 5, the sh-oligopeptide-1 comprises a sequence at least 90%, 95%, or 98% identical to SEQ ID NO 11, the sh-Polypeptide-4 comprises a sequence at least 90%, 95%, or 98% identical to SEQ ID NO 12, and the Acetyl sh-Oligopeptide-77 Amide comprises a sequence at least 90%, 95%, or 98% identical to SEQ ID NO 13, with each growth factor being present at a concentration ranging from 0.001 wt % to 1 wt % of the overall composition and more preferably from 0.009 wt % to 1 wt %.

This composition further includes hyaluronic acid (CAS No. 9067-32-7) and silanol and/or organosilanol (CAS No.: 4253-34-3) is methylsilanetriol (CAS No. 2445-53-6), at substantially the same concentrations as those previously discussed above and for reasons similar to those disclosed above as well as for the scalp rejuvenation and restore hair growth purposes disclosed herein for this specific composition.

This composition may be used alone or in conjunction with the other compositions/formulations disclosed herein as well as with microneedling procedures as disclosed below in the “Methods of Use”. An exemplary reparative composition/formulation is shown immediately below within Table 4.

TABLE 4
HAIR/SCALP COMPOSITION

| No | IGREDIENTS | CAS | R [symbol] % | FUNCTION |
|---|---|---|---|---|
| 1 | Water (Aqua) | 77 [symbol] - [symbol] | [symbol] .000 | Solvent |
| 2 | B [symbol] Alcohol (and) D [symbol] Acid (and) Water | 100- [symbol] 1-6 [symbol] 520-46-6 [symbol] 77 [symbol] 2-18- [symbol] | 1.000 | Preservative |
| 3 | H [symbol] Acid (and) [symbol] (and) Water (and) Butylene Glycol | [symbol] 0 [symbol] -7, [symbol] 253- [symbol] - [symbol] 7732-18- [symbol] [symbol] 07- [symbol] -0 | 1.000 | M [symbol] ing |
| 4 | Water (and) P [symbol] yle [symbol] Glycol (and) 1,2-hexanediol (and) Sodium Phosphate | [symbol] 322- [symbol] - [symbol] | 1.000 | Surfactant, |
| | (and) Lecithin (and) sh-Oli [symbol] -2 (and) sh-Polypeptide- [symbol] (and) sh- | | | Growth |
| | Polypeptide-11 (and) sh-Polypeptide-1 (and) sh-Polypeptide-3 (and) sh- | | | Factors |
| | Polypeptide- [symbol] (and) sh-Polypeptide- [symbol] (and) sh-Polypeptide-10 (and) sh- | | | |
| | Polypeptide- [symbol] (and) Ace [symbol] sh-O [symbol] Amide (and) A [symbol] O [symbol] peptide-17 | | | |
| | Amide | | | |

[symbol]

indicates data missing or illegible when filed

Kits

In certain aspects, each of the compositions/formulations disclosed herein are preferably sterile. These compositions/formulations may be further packaged within single use vials and/or containers (single use disposable vials and/or disposable containers), or alternatively may be packaged within multi-use vials and/or containers. In certain aspects, any one of the above mentioned compositions/formulations may be packaged alone within a kit. Alternatively, any one of the above compositions/formulations may be packaged within a kit that further includes microneedles. The number of microneedles provided in the kit sufficiently corresponds with the composition amount packaged within the kit.

In certain aspects, the kit may include at least two compositions selected from the regenerative composition/formulation, the reparative composition/formulation, and the hair/scalp composition/formulation as disclosed above. In this aspect, the kit may further include microneedles that sufficiently correspond with the composition amount included within the kit in which the microneedles may be further used with the compositions as disclosed herein (e.g., as disclosed within the “Methods of Use” below).

In certain additional aspects, the kit may include all three of the regenerative composition/formulation, reparative composition/formulation, and the hair/scalp composition/formulation as disclosed above, and the kit may further include microneedles that sufficiently correspond with the composition amount included within the kit and that may be further used with the compositions as disclosed herein (e.g., as disclosed within the “Methods of Use” below).

Methods of Use

Also disclosed is a method of reducing wrinkles and/or fine lines over a predetermined time period, the method comprises (a) applying on the skin of a subject at least one of the compositions disclosed above; and (b) repeating the application of the composition to the skin of the subject at predetermined time periods (e.g., once a day or twice a day) thereby reducing wrinkles, fine lines, and/or the appearance thereof during the predetermined time period. In certain aspects, the compositions are applied twice a day (e.g., morning and at night). In certain aspects, this method further comprises: before step (a) (e.g., either immediately before or within 5 to 10 minutes before) performing a microneedling procedure on the subject to increase delivery efficacy of the composition to the subject. In certain aspects, this method further comprises after step (a) and before step (b) (e.g., either immediately before or within 5 to 10 minutes before) performing a microneedling procedure on the subject to increase delivery efficacy of the composition to the subject. In certain aspects, the microneedling procedure is performed at least once per week but no more than twice per week depending on whether the subject can tolerate this procedure twice per week.

Also disclosed is a method for reducing pore size over a predetermined time period, the method comprises (a) applying on the skin of a subject at least one of the compositions disclosed above; and (b) repeating the application of the composition to the skin of the subject at predetermined time periods (e.g., once a day or twice a day) thereby reducing pore size and/or the appearance thereof during the predetermined time period. In certain aspects, the compositions are applied twice a day (e.g., morning and at night). In certain aspects, this method further comprises before step (a) (e.g., either immediately before or within 5 to 10 minutes before) performing a microneedling procedure on the subject to increase delivery efficacy of the composition to the subject. In certain aspects, this method further comprises after step (a) and before step (b) (e.g., either immediately before or within 5 to 10 minutes before) performing a microneedling procedure on the subject to increase delivery efficacy of the composition to the subject. In certain aspects, the microneedling procedure is performed at least once per week but no more than twice per week depending on whether the subject can tolerate this procedure twice per week.

Also disclosed is a method of enhancing skin texture over a predetermined time period, the method comprises (a) applying on the skin of a subject at least one of the compositions disclosed above; and (b) repeating the application of the composition to the skin of the subject at predetermined time periods thereby improving and/or enhancing skin texture and/or the appearance thereof during the predetermined time period. In certain aspects, the compositions are applied twice a day (e.g., morning and at night). In certain aspects, this method further comprises before step (a) (e.g., either immediately before or within 5 to 10 minutes before) performing a microneedling procedure on the subject to increase delivery efficacy of the composition to the subject. In certain aspects, this method further comprises after step (a) and before step (b) (e.g., either immediately before or within 5 to 10 minutes before) performing a microneedling procedure on the subject to increase delivery efficacy of the composition to the subject. In certain aspects, the microneedling procedure is performed at least once per week but no more than twice per week depending on whether the subject can tolerate this procedure twice per week. In certain aspects, a user's scalp appearance may be rejuvenated and/or a user's hair may be re-grown by applying the hair composition/formulation disclosed above to a user's scalp and using substantially the same steps and procedures as those disclosed immediately above within the “Methods of Use” section (for reducing wrinkles, reducing pore size, and improving skin texture respectively).

### Working Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for.

Cell Culture Data Comparing Proliferative Effects Of One Recombinant Human Growth Factor Versus A Combination Of Ten Recombinant Human Growth Factors (Proof of Concept)

| Name | Test to Ascertain Cell Proliferation Rate |
|---|---|
| Date | Jan. 15, 2020-Feb. 7, 2020 |
| Tested Material | Facial Rejuvenation, EGF |
| Department in | PnP Bipharm Co., Ltd., R&D-Culture Expert |
| Charge | Team on behalf of SkinGen International |

| | Reagent | Manufacturer | Catalog Number |
|---|---|---|---|
| 1 | DMEM high glucose | Hyclone | SH30243.01 |
| 2 | Penicillin-Streptomycin | Hyclone | SV30079.01 |
| 3 | Fetal bovine serum | Lonza | 14-501F |
| 4 | Bovine serum albumin | Sigma | A7638-1G |
| 5 | PBS without Ca, Mg | Welgene | LB001-02 |
| 6 | Trypan blue | Sigma | T8154 |
| 7 | Trypsin-EDTA | Hyclone | SH3004202 |
| 8 | Cell counting kit-8 | Dojindo | KR833 |

Formulating Solution(s)

### 1. Formulation of DMEM High Glucose Complete Culture Medium

A. After formulation, aliquot into 50 mL conical tube and store at 4° C.

DMEM high glucose culture media

| | | |
|---|---|---|
| DMEM high glucose medium | 450 | ml |
| FBS (fetal bovine serum) | 50 | ml |
| Penicillin-streptomycin | 5 | ml |

DMEM high glucose test media

| | | |
|---|---|---|
| DMEM high glucose medium | 450 | ml |
| BSA | 0.25 | g |
| Penicillin-streptomycin | 5 | ml |

CCK-8 test media

| | | |
|---|---|---|
| DMEM high glucose medium | 100 | ml |
| Penicillin-Streptomycin | 1 | ml |
| Cell counting kit-8 | 10 | ml |

Test Procedure:

1. Culture Balb/c-3T3 cell with 10% FBS in DMEM high glucose culture media onto 75U-flask. Proceed subculture when flask reaches 80-90% confluency.
2. Cell seeding (Day #1)
A. Remove Balb/c-3T3 cells from the 75U-flask. Decant as much supernatant as possible.
B. Detaching attached cells after washing by adding D-PBS.
C. Adding Trypsin-EDTA uniformly over the surface of the cell culture in 75U-Flask.
D. Incubate the cells in 37° C., 5% CO₂ incubator for 1 minute.
E. Use a microscope to ensure Balb/c-3T3 cells are detached, then add 20 ml of fresh culture media and detached Balb/c-3T3 cells into a 50 ml tube.
F. Centrifuge at 1000 rpm for 3 minutes.
G. Remove the supernatant, then add 5-7 ml of media and resuspend.
H. Count the number of cells using the Hemocytometer.
I. Formulate a cell suspension liquid with 3.0×10⁵ cells/15 ml, then culture onto the 75U-flask.

Formulate a cell suspension liquid with 2.0×10⁴ cells/0.1 ml per well. Move onto a 96well plate and fill the empty wells with 200 pl of PBS. Incubate for 24 hours. (37° C., 5% CO₂ incubator)

### 3. Starvation (Day #2)

A. Check the Balb/c-3T3 cells in the 96 well plate with a microscope. Use a multi-pipette to remove supernatant.
B. Add 100 pl of D-PBS per well to wash by shaking lightly (2-3 times). Use a multi-pipette to completely remove D-PBS.
C. Add 100 pl of DMEM high glucose test media per well and culture overnight. (37° C., 5% CO₂ incubator)
   4. Application of growth factor (Day #3)
A. After starvation (approx. after 20 hours), treat growth factor with serial dilution according to concentration.
B. Culture for 24 hours (37° C., 5% CO₂ incubator).

### 5. Application of CCK-8 (Day #4)

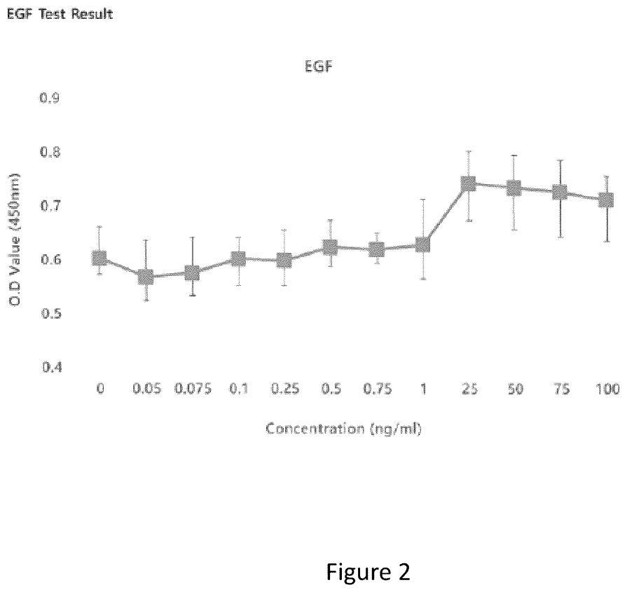

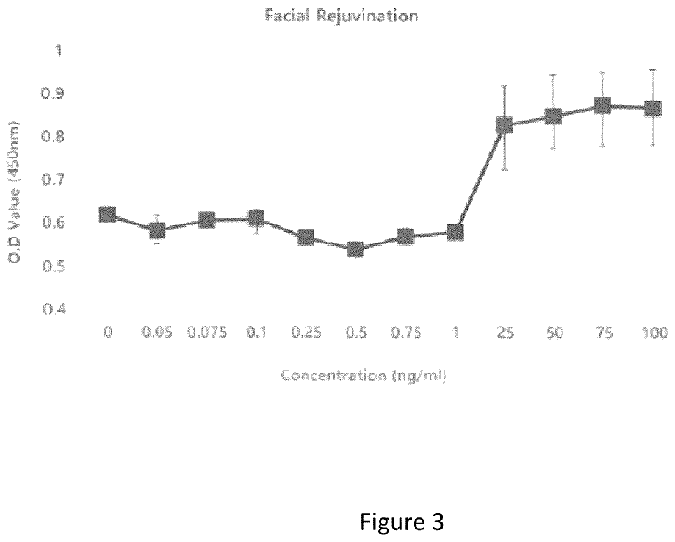

A. Take out the 96 well plate after incubating for 24 hours. Use a multi pipette to remove all the media out of the 96 well plate then add 100p1 ofcck-8 test media per well.
B. Culture for 3 hours (37° C., 5% C02 incubator) then measured the optical density by using a micro plate at 450 nm.
   6. Results: FIGS. 2-4 depict cell proliferation results with cells respectively treated with EGF alone (i.e., a single recombinant human growth factor), the formulation having ten recombinant human growth factors, and a comparison of optical density measurements between the cells treated with EGF alone (i.e., a single recombinant human growth factor) versus the formulation having ten recombinant human growth factors. As shown in FIG. 4, cells treated with the formulation having ten recombinant human growth factors exhibited a much greater optical density over the same time period, thus indicating greater cellular proliferation occurs within cells having been contacted/treated with a formulation having ten recombinant human growth factors in view of cells contacted/treated with a single growth factor and further evidencing proof of concept.

Clinical Tests and Results

A study was conducted to evaluate the efficacy and safety of an exemplary topical/transdermal composition having recombinant human growth factors as disclosed herein and in conjunction with a micro needling procedure to evaluate efficacy for anti-aging and anti-wrinkle benefits in healthy female subjects.

Primary Objectives:

To evaluate the reduction in fine lines and wrinkles defined time points in comparison to the baseline (i.e., pre-treatment).

To evaluate the improvement in evenness of skin tone, skin clarity and age spots.

To evaluate improved skin texture and improved skin elasticity/firmness.

Secondary Objectives:

To evaluate skin hydration.

To evaluate the product safety by skin tolerance evaluation performed by dermatologist.

Assessment Parameters:

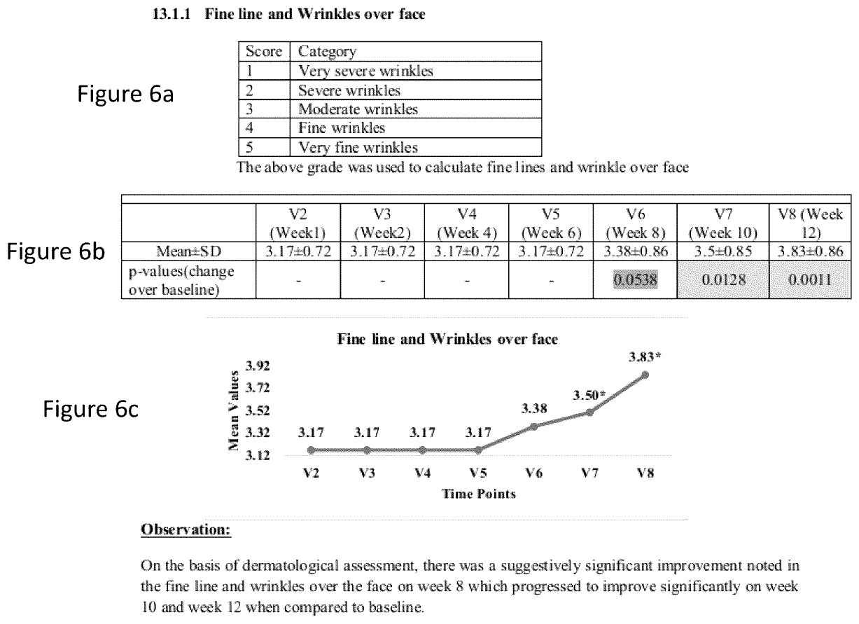

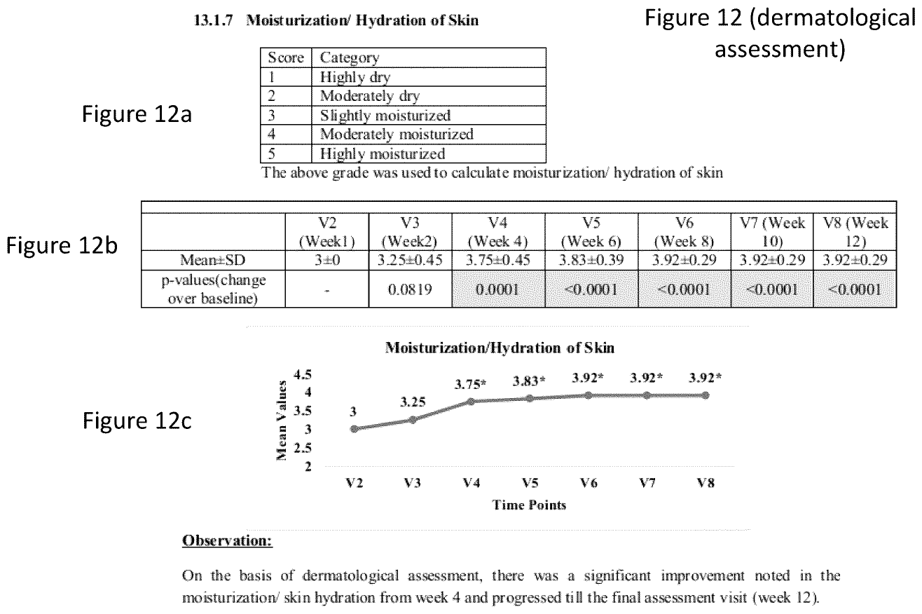

The Dermatologists' assessment for fine lines and wrinkles and the overall improvement in skin texture, age spots and pigmentation and hydration level are shown in FIGS. 6a-12c.

Subject self-assessment (not shown) for perceived improvement in fine lines and wrinkles, overall improvement in age spots and pigmentation, skin texture and moisturization.

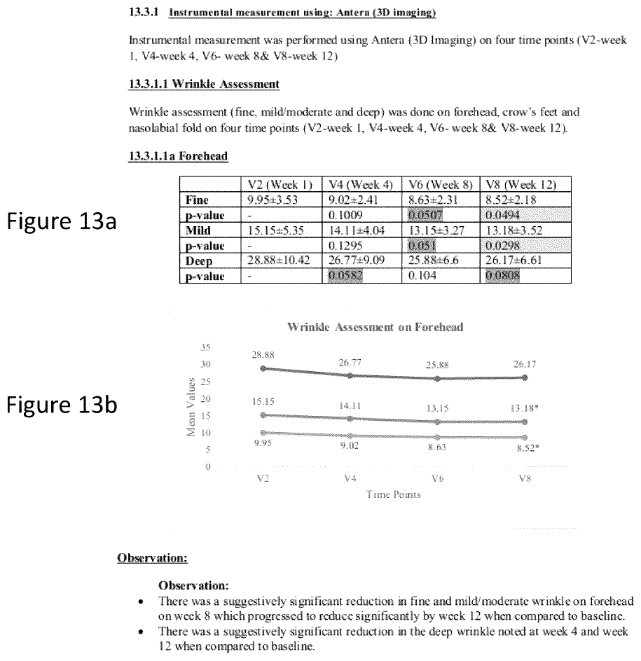

Instrumental measurements and assessments using: Antera (3D imaging) are shown in FIGS. 13a-24e.

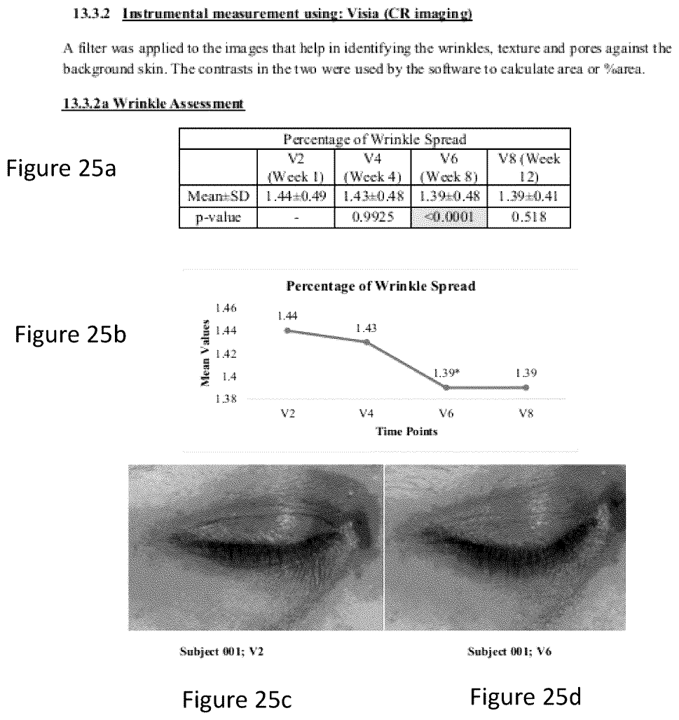

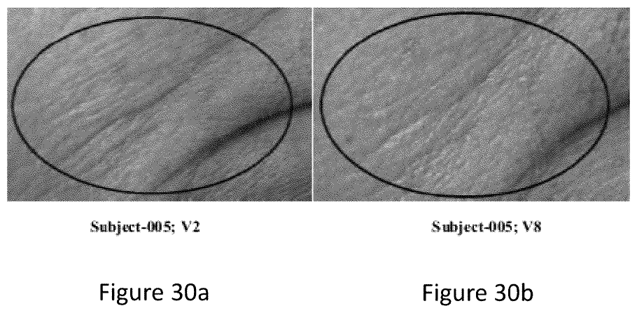

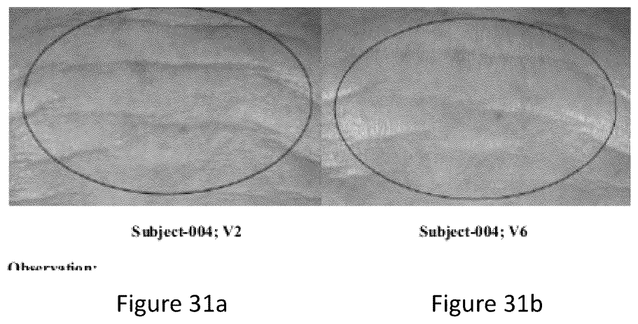

Image based comparative assessments using VISIA images (CR imaging) are shown in FIGS. 25a-31c.

Selection Criteria:

A total of twelve female subjects (as shown in the Table immediately below) were required to complete the study. Female subject falling within the age group of 35-60 and Fitzpatrick skin types III-V were required for the study along with having aging concerns and/or signs of aging. Nineteen subjects were initially screened and enrolled after satisfying inclusion and exclusion criteria. Seventeen subjects were used in the study, which are as follows:

| | | | FitzpatrickSkin |
|---|---|---|---|
| Sub ID | Age | Gender | Type |
| S01 | 49 | F | IV |
| S02 | 44 | F | IV |
| S03 | 51 | F | IV |
| S04 | 61 | F | IV |
| S05 | 51 | F | IV |
| S06 | 46 | F | IV |
| S07 | 50 | F | IV |
| S08 | 59 | F | IV |
| S09 | 56 | F | III |
| S10 | 57 | F | IV |
| S11 | 49 | F | IV |
| S12 | 49 | F | IV |
| S13 | 50 | F | IV |
| S14 | 50 | F | IV |
| S15 | 43 | F | IV |
| S16 | 48 | F | IV |
| S17 | 48 | F | IV |

Twelve subjects completed the study. Two subjects dropped out of the study due to adverse events (dry and/or inflamed skin) while three additional subjects were dropped from the study due to failure to follow-up and/or maintain the below described protocol.

Products Used:

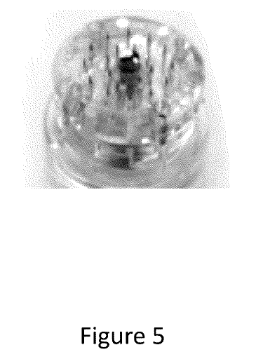

1 mm micro needles (Hydra 20 Micro Needle as shown in FIG. 5) were used by dermatologist during in office visits in conjunction with the exemplary topical/transdermal composition. 0.5/0.6 mm micro needles were provided to the subjects for at home use in conjunction with the exemplary topical/transdermal composition.

Study Outline:

There were a total of 8 visits:
Visit 1-day 0—Screening, enrolment and baseline visit
Visit 2-Week 1
Visit 3-Week 2
Visit 4-Week 4
Visit 5-Week 6
Visit 6-Week 8
Visit 7-Week 10
Visit 8-Week 12—Final assessment visit

The study was conducted for a period of approximately 3 months for each subject and included a total of 8 visits i.e., visit 1 (day 0, screening, and enrollment assessment visit) and on visits 2, 3, 4, 5, 6 and 7 all assessments were performed followed by micro-needling procedure. Visit 8 was the final assessment visit; there was no procedure on this assessment visit.

A wash out period of 7 days and preparatory period of 2 days was maintained between visit 1 and visit 2. A sufficient number of healthy female subjects of 35-60 (inclusive both ages) years of age was screened, so that total 10 subjects complete Part I (exploratory pilot study) of the study. The subjects who satisfied inclusion and exclusion criteria were enrolled after obtaining informed consent.

Prior to the baseline assessment visit, there was a wash out period of 1 week. During their wash out phase the subjects received a skin care kit containing cleanser, day cream (with sunscreen) and a night cream (with moisturizer). The last two days of this period was regarded as the preparatory phase of the study during which the subjects stopped the day cream and night cream.

First anti-aging hydra needle procedure was performed at the site by a trained designee on visit 2, after baseline assessment. On visits 3, 4, 5, 6 and 7 subjects underwent dermatological assessment; subject self-assessment for perceived improvement, safety assessment, and instrumental assessment followed by anti-aging hydra needle procedure.

On the days when the subjects were not visiting the study center, they underwent Anti-aging hydra needle procedure by trained personnel appointed by the study center at their home every alternate day except on Sundays.

| Visits | V1 | | V2 | V2 | V3 | V4 | V5 | V6 | V7 | V8 |
|---|---|---|---|---|---|---|---|---|---|---|
| Time points | Screening | | T0 | T1 | Week 02 | Week 04 | Week 06 | Week 08 | Week 10 | Week 12 |
| Dermatological | — | Washout | √ | √ | √ | √ | √ | √ | √ | √ |
| assessment | | period | | | | | | | | |
| VISIA CR Imaging | — | ϕpreparatory | √ | √ | — | √ | — | √ | — | √ |
| Antera (3DImaging) | — | phase | √ | √ | — | √ | — | √ | — | √ |
| Imaging assessment | — | | √ | √ | — | √ | — | √ | — | √ |

At Site (by Investigator)

Subjects visited the site every two weeks. The micro needling procedure was performed (using 1 mm needle) after topical anesthetic cream application. The anesthetic cream was applied in thin film without occlusion. Following the micro needling procedure, the exemplary topical/transdermal composition was applied to the subject on and around the site of micro needling.

At Home (self)—Twice a week

Procedure was performed by the subject (self) using 0.5/0.6 mm needle.

Subject visited the site at day 3 for training.

First self-procedure was done at the site under the supervision of the investigator.

The composition was applied immediately after the procedure and not 12 hrs prior to the procedure.

Procedure was performed in the late evening.

Data Processing and Statistical Methodology

As discussed further below, data was compiled using multiple different assessments (i.e., dermatological assessment, subject-self assessment, and multiple different image assessments) to obtain statistical data regarding whether the exemplary compositions exhibited advantageous effects during the study while concurrently minimizing subjective bias via the use of these multiple different assessment techniques.

Data was captured in electronic case report form using a mobile app built by MSCR. Different types of edit check were applied to avoid discrepancy while entering the data. After completion of the study, data was exported into Excel® and then the data was checked again as a process of data validation. After completion of data validation, the data was locked for analysis.

For Efficacy—The data of all subjects who successfully completed the study was considered for the statistical analysis. Data of 12 subjects was considered for the statistical analysis.

Product Safety—The data of all the subjects available on each assessment visit was considered which included lost to follow up, drop outs till their last participation visit.

Descriptive statistical analysis was carried out in the present study. In all analysis, a 2-sided significance level of 5% (p-value<0.05) was used to determine significance levels.

Results on continuous measurements were presented on Mean±SD and results on categorical measurements were presented in Number (%). Significance was assessed at 5% level of significance. Paired t-test was performed to find out efficacy in comparison to baseline on continuous scales such as instrumental assessments.

Statistical figures:
Significant 5%=highlighted in yellow
Suggestive of significance at 10%=highlighted in blue

Statistical software: The Statistical software, R-ver.3.1.2 was used for the analysis of the data and Microsoft word and Excel have been used to generate graphs, tables etc.

Dermatological Assessment:

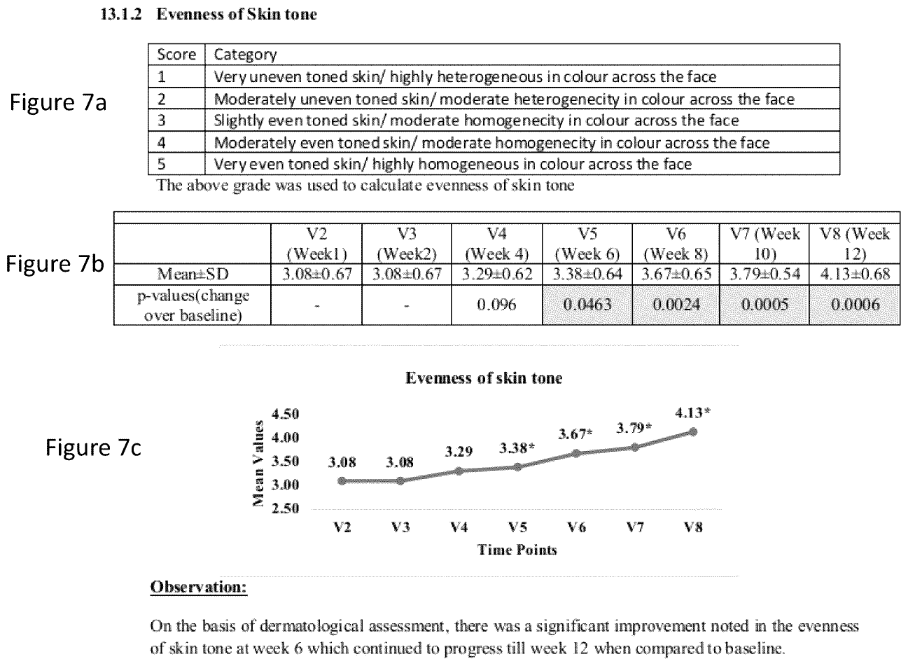

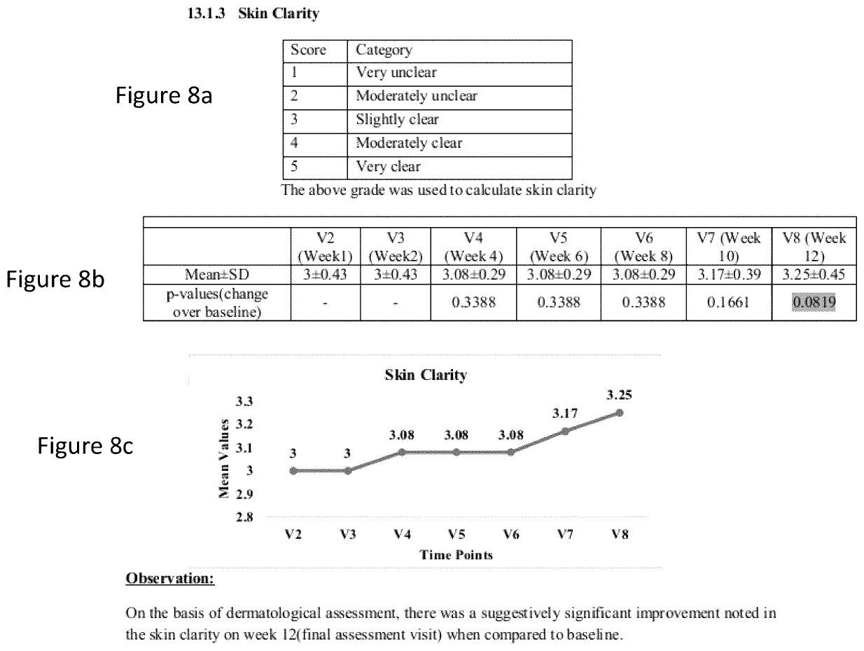

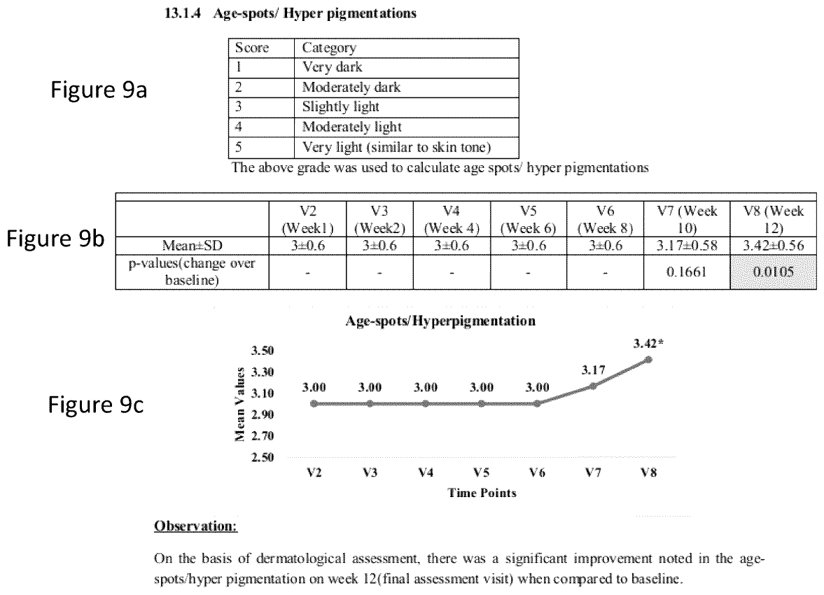

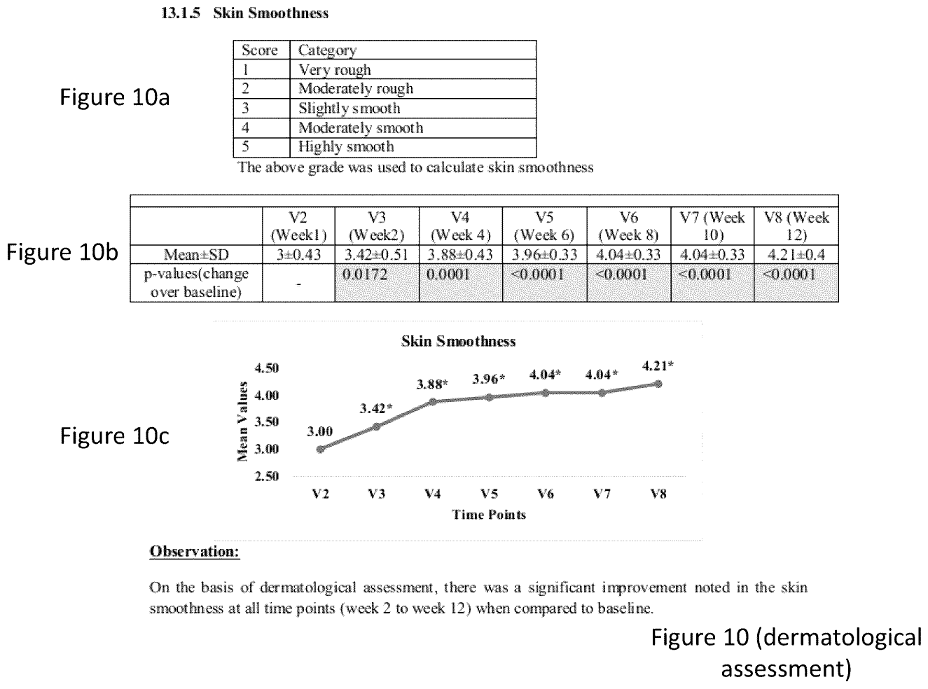

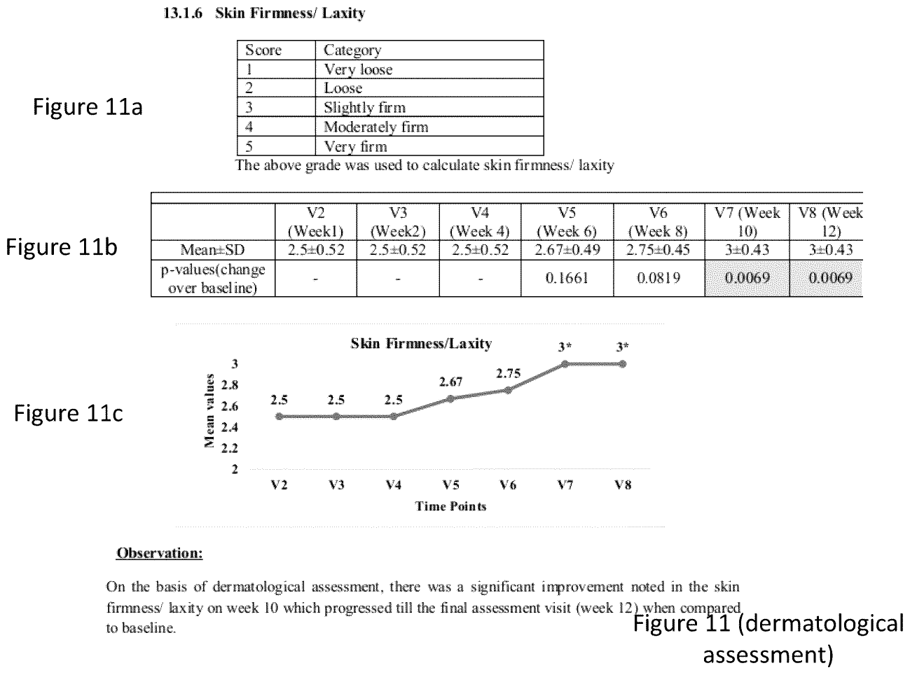

Fine line(s) and wrinkles (FIGS. 6a-6c), evenness of skin tone (FIGS. 7a-7c), age spots (FIGS. 9a-9c), skin smoothness (FIGS. 10a-10c), and skin firmness and hydration (FIGS. 11a-11c) of skin was noted to show a statistically significant improvement when compared to baseline (i.e., Week 12 (V8) versus Week 1 (V1)). There was a suggestively significant improvement noted for skin clarity (FIGS. 8a-8c) on final assessment visit (week 12).

Subject Self-Assessment (Data Not Shown):

There was a significant improvement perceived for all the six parameters (fine lines and wrinkles, evenness of skin tone, skin clarity, age spots, skin firmness and hydration of skin) when compared to baseline (data not shown).

Instrumental measurement using Antera (3D Imaging)

(FIGS. 13a-24e):

Wrinkle Assessment: Mild to moderate wrinkle on the forehead was noted to reduce significantly by week 12 (final assessment visit; FIGS. 13a, 13b, 14c, and 14d respectively). Deep wrinkles were noted to reduce (suggestively significant) by week 4 when compared to baseline (FIGS. 13a, 13b, 14e, and 14f). There was a suggestively significant reduction in fine wrinkle on forehead at week 8 which progressed to significantly reduce by week 12 (final assessment visit) when compared to baseline (FIGS. 13a, 13b, 14a, and 14b).

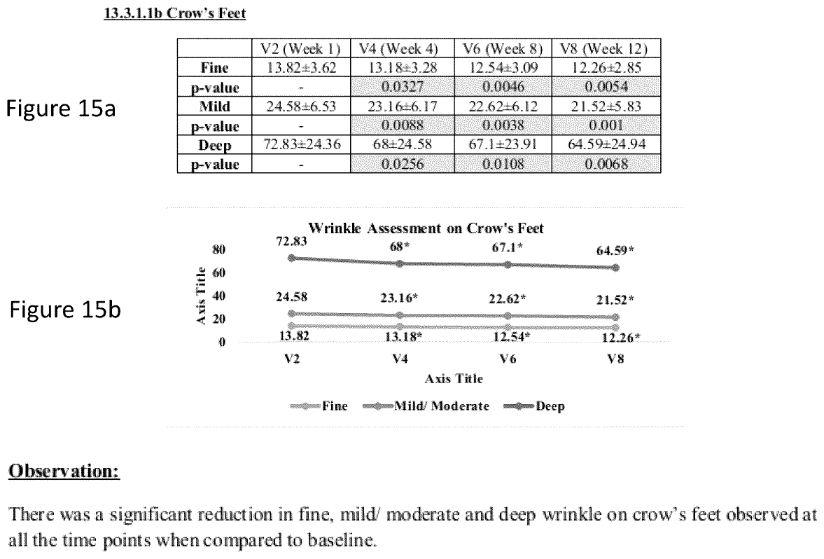

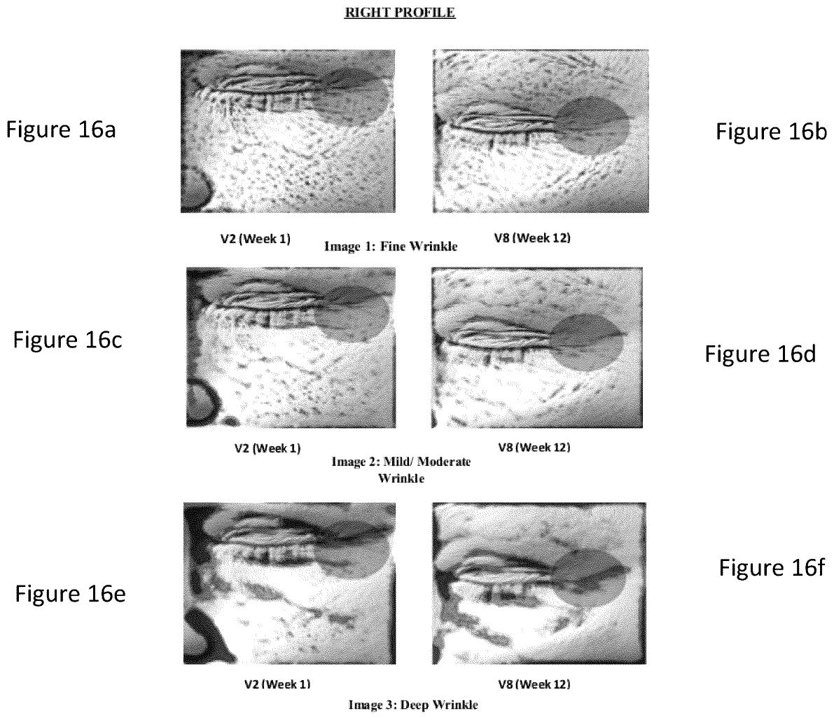

There was a significant reduction in wrinkles (fine, mild and deep) on Crow's feet observed at all the given time points when compared to baseline (FIGS. 15a-16f).

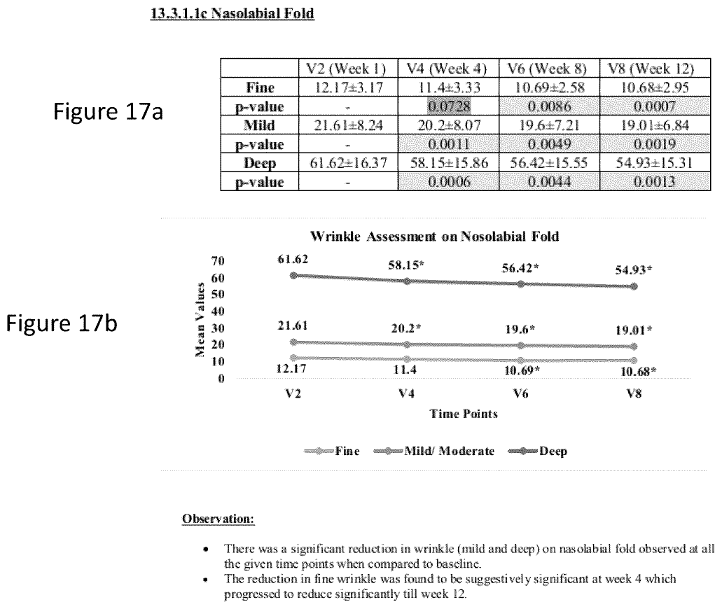

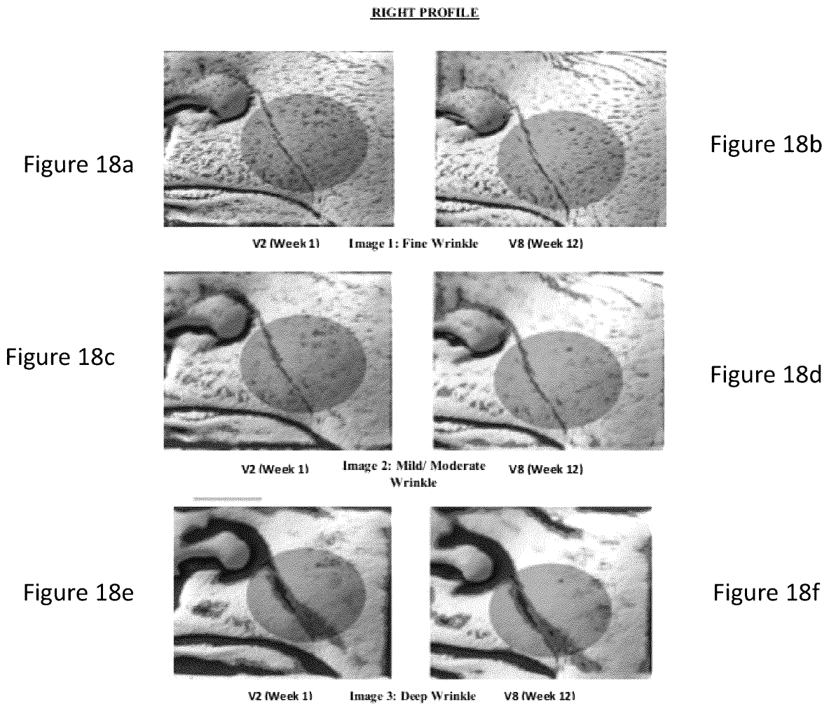

There was a significant reduction in wrinkles (mild and deep) on nasolabial fold which was observed at all the given time points when compared to baseline (FIGS. 17a, 17b, and 18c-18f). The reduction in fine wrinkles was found to be suggestively significant at week 4 which progressed to be significant at week 8 and week 12 (FIGS. 17a, 17b, 18a, and 18b).

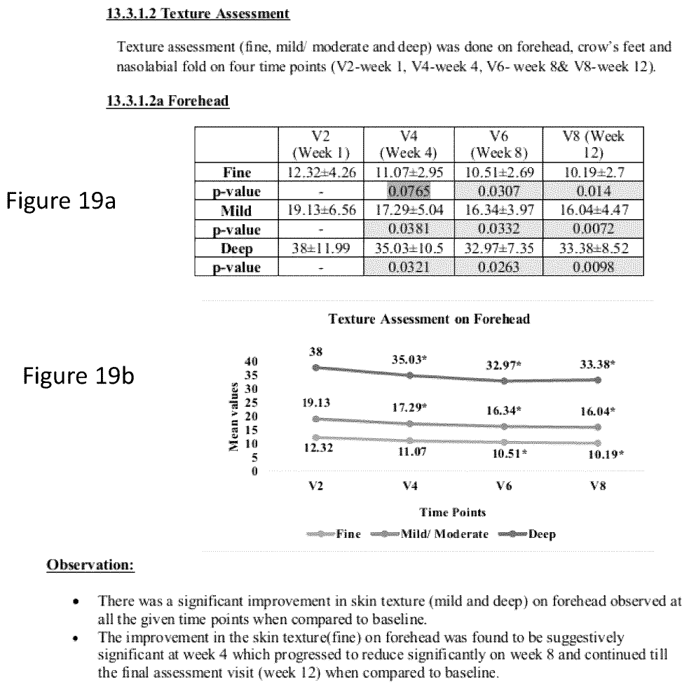

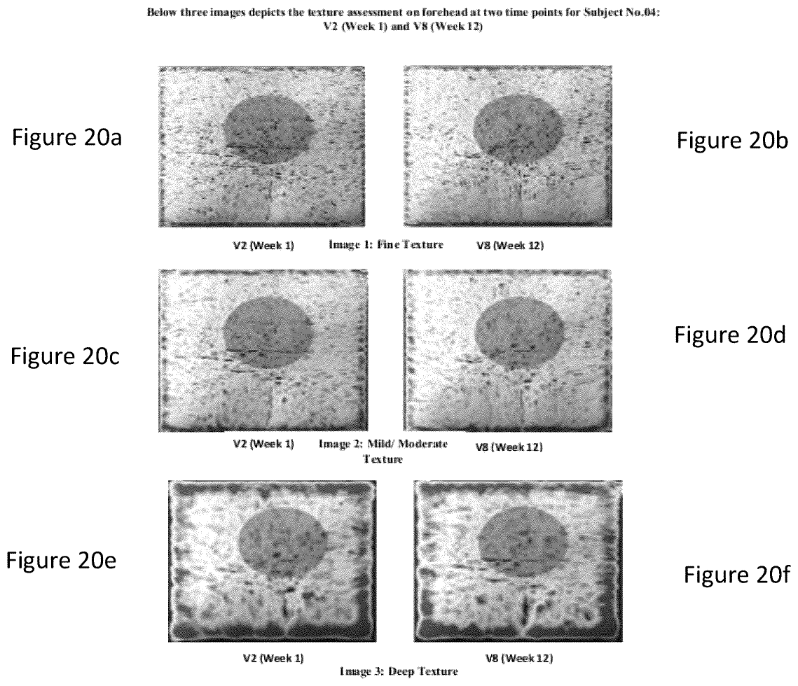

Texture Assessment: There was a significant improvement in skin texture (mild and deep) on forehead which observed at all the given time points when compared to baseline (FIGS. 19a, 19b, and 20c-20f). The improvement in the fine skin texture on forehead was found to be suggestively significant at week 4 which progressed to be significant by week 8 and continued till the final assessment visit (week 12) when compared to baseline (FIGS. 19a, 19b, 20a, and 20b).

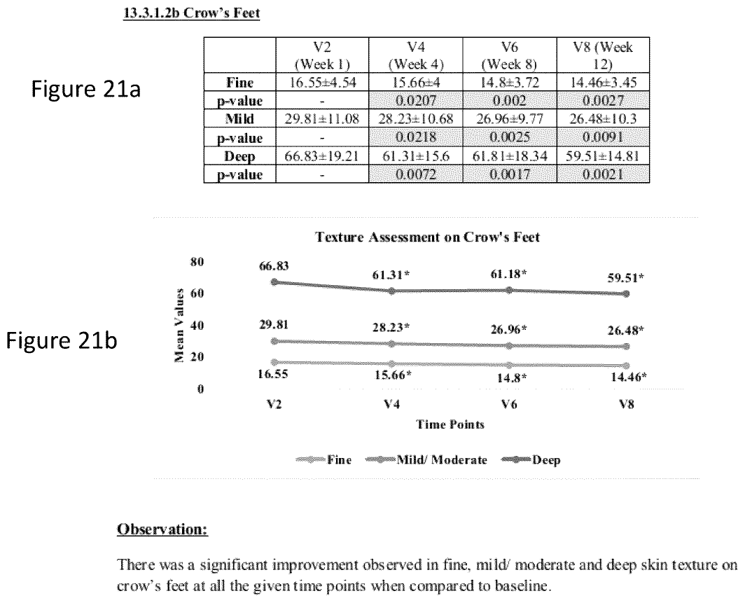

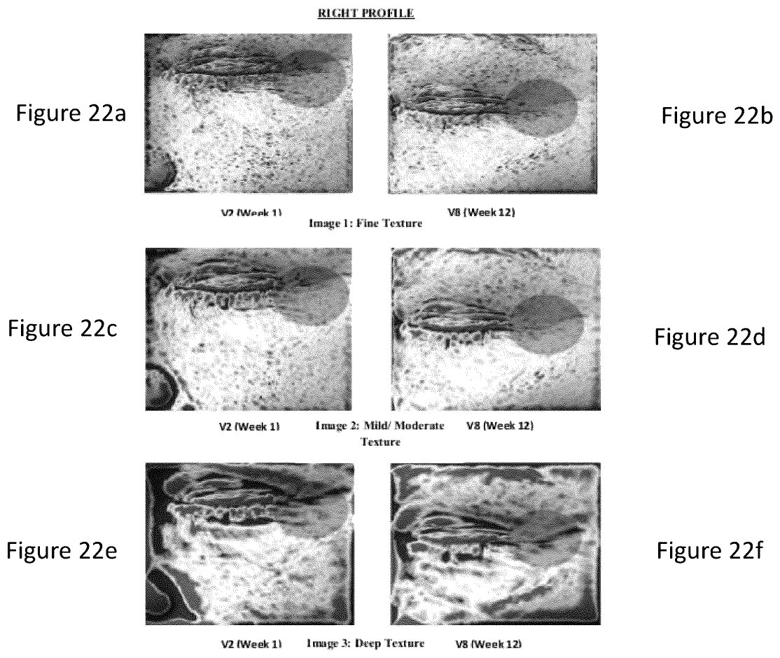

Significant improvement in texture (fine, mild and deep) on Crow's feet was observed at all the given time points when compared to baseline (FIGS. 21a, 21b, and 22c-22f).

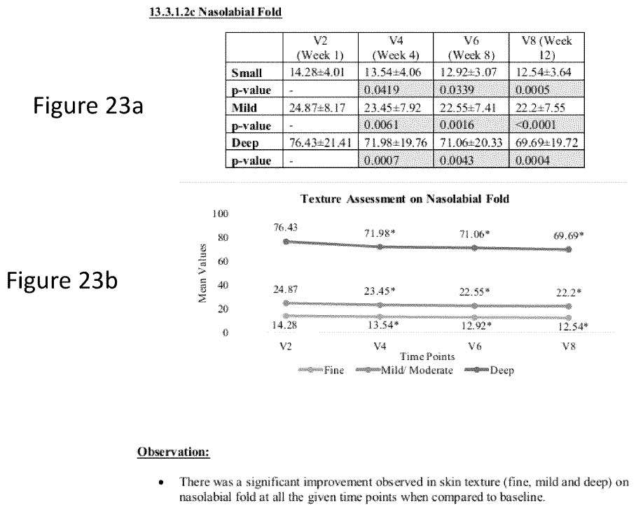

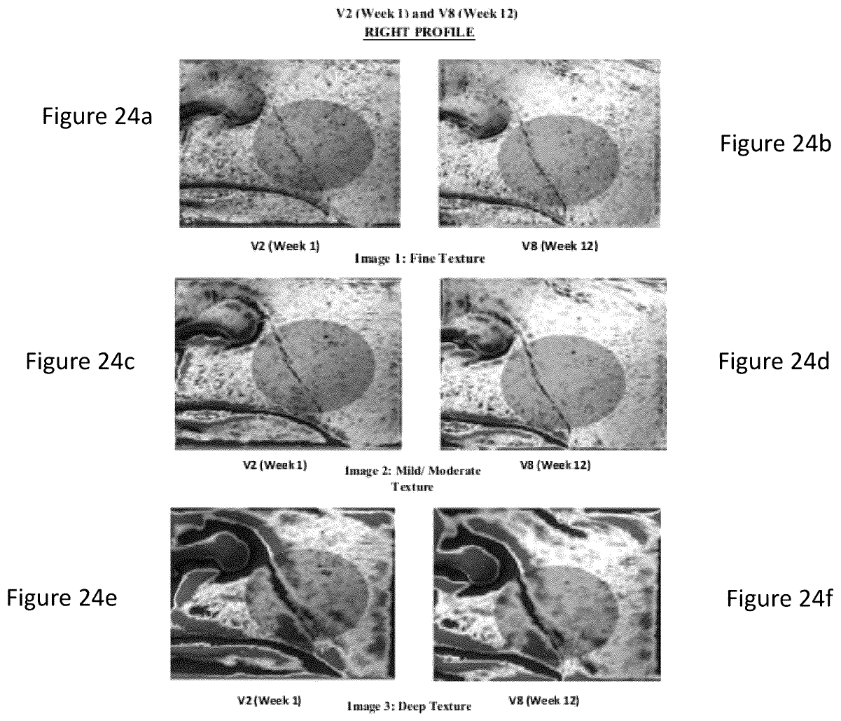

Significant improvement in skin texture (mild and deep) on nasolabial fold was observed at all the given time points when compared to baseline (FIGS. 23a, 23b, and 24c-24f). The improvement in the fine skin texture on nasolabial fold was found to be suggestively significant at week 4 which progressed to be significant by week 8 and continued till the final assessment visit (week 12) when compared to baseline (FIGS. 23a, 23b, 24a, and 24b).

Instrumental measurement using Visia (CR Imaging):

Wrinkle Assessment: There was a significant decrease in the percentage of wrinkles that was observed at a given test site on week 8 when compared to week 1 (FIGS. 25a-25d).

Texture Assessment: There was a significant improvement noted in the percentage of texture of the skin on three given time point (week 4 to week 12) when compared to baseline (FIGS. 26a-26d).

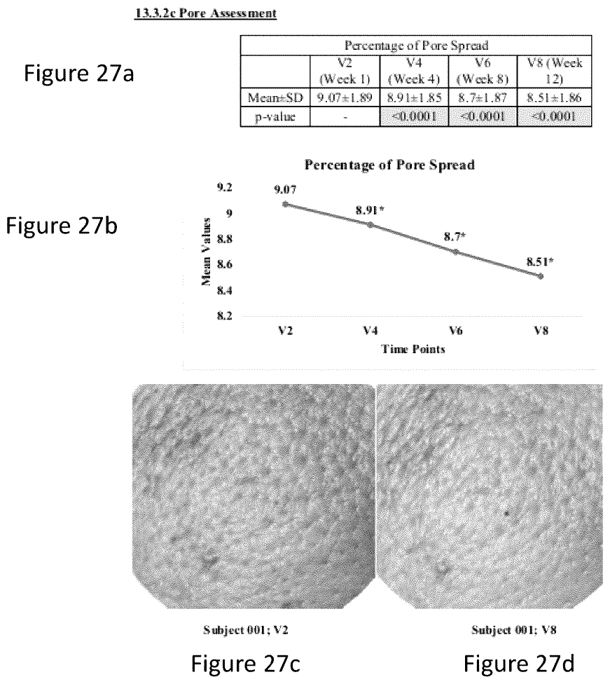

Pore Assessment: There was a significant decrease noted in the percentage of pore spread at a given test site on all the three visit (week 4 to week 12) when compared to baseline (FIGS. 27a-27d).

Image Based Comparative Assessment:

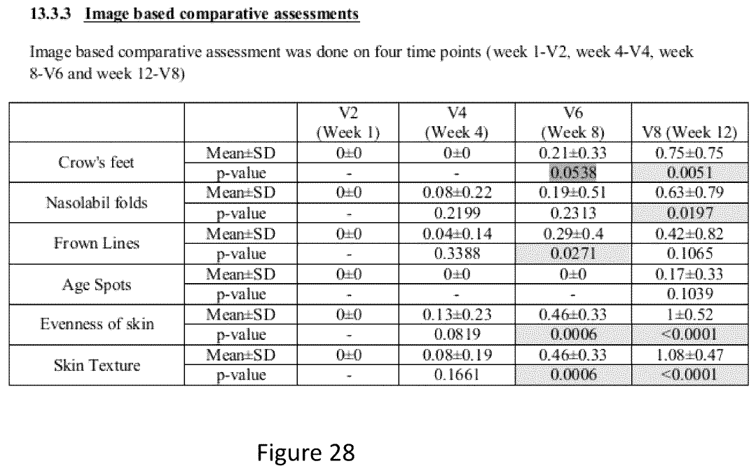

There was a significant reduction of wrinkles observed at Crow's feet, Nasolabial folds and Frown lines (FIG. 28 and FIGS. 29a-31b) when compared to baseline.

There was no significant change observed in the age spots, evenness of skin and skin texture when compared to baseline.

### Conclusion(s)

The study was conducted to evaluate the efficacy and safety of skin care regimen in adjunction with the micro needling procedure for providing anti-ageing and anti-wrinkle benefits in healthy female subjects.

As per the dermatological evaluations, there was significant improvement in Fine line & wrinkles, evenness of skin tone, age spots, skin smoothness, skin firmness and hydration of skin when compared to baseline. There was suggestively significant improvement noted for skin clarity on final assessment visit (week 12).

As per the subject self-assessment, there was a significant improvement noted for all the six parameters (fine line & wrinkles, evenness of skin tone, skin clarity, age spots, skin firmness and hydration of skin) at time points (week 2 to week 12) when compared to baseline.

As per instrumental assessment (Antera (3D Imaging)).

Wrinkle: There was a significant reduction in mild and fine wrinkles at week 12 (final assessment visit) observed on all the tested sites namely, forehead, crow's feet and nasolabial fold when compared to baseline. There was significant reduction in deep wrinkle for test sites (crow's feet and nasolabial fold) at week 12 (final assessment visit) when compared to baseline and suggestive reduction for test site (Forehead) at week 12 when compared to baseline.

Texture: There was a significant improvement observed in skin texture (mild and deep) on forehead, crow's feet and nasolabial fold at all the given time points when compared to baseline. The improvement in the fine skin texture on crow's feet and nasolabial fold was found to be significant at all-time points when compared to baseline however the improvement in the fine skin texture on forehead was found to be suggestively significant at week 4 when compared to baseline.

As per instrumental assessment Visia (CR Imaging), a significant reduction in the percentage of wrinkles was observed at week 8 when compared to week 1. Also, there was a significant improvement noted in the percentage of texture & pore spread on three given time points (week 4, week 8 and week 12) when compared to baseline.

As per image based comparative assessment, a significant reduction of wrinkle was observed at Crow's feet, Nasolabial folds and Frown lines, evenness of skin and skin texture at week 12 when compared to baseline. However, there was no change noted in the age spots when compared to baseline.

Product Safety: Two subjects (S02 and S06) could not tolerate the Micro needling procedure and developed dry and irritable skin post first procedure. As per the dermatologists opinion this may be a result of severe skin dryness in combination with topical anesthetic product. The protocol was modified accordingly and was thereafter well tolerated by rest of the subjects who participated in the study. Subjects 02 and 06 were followed-up till complete resolution of skin reaction which resolved with.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

SEQ ID NO 1:
Gln Arg Lys Arg Arg Asn Thr Ile His Glu Phe Lys Lys Ser Ala Lys
Thr Thr Leu Ile Lys Ile Asp Pro Ala Leu Lys Ile Lys Thr Lys Lys
Val Asn Thr Ala Asp Gln Cys Ala Asn Arg Cys Thr Arg Asn Lys Gly
Leu Pro Phe Thr Cys Lys Ala Phe Val Phe Asp Lys Ala Arg Lys Gln
Cys Leu Trp Phe Pro Phe Asn Ser Met Ser Ser Gly Val Lys Lys Glu
Phe Gly His Glu Phe Asp Leu Tyr Glu Asn Lys Asp Tyr Ile Arg Asn
Cys Ile Ile Gly Lys Gly Arg Ser Tyr Lys Gly Thr Val Ser Ile Thr
Lys Ser Gly Ile Lys Cys Gln Pro Trp Ser Ser Met Ile Pro His Glu
His Ser Phe Leu Pro Ser Ser Tyr Arg Gly Lys Asp Leu Gln Glu Asn
Tyr Cys Arg Asn Pro Arg Gly Glu Glu Gly Gly Pro Trp Cys Phe Thr
Ser Asn Pro Glu Val Arg Tyr Glu Val Cys Asp Ile Pro Gln Cys Ser
Glu Val Glu Cys Met Thr Cys Asn Gly Glu Ser Tyr Arg Gly Leu Met
Asp His Thr Glu Ser Gly Lys Ile Cys Gln Arg Trp Asp His Gln Thr
Pro His Arg His Lys Phe Leu Pro Glu Arg Tyr Pro Asp Lys Gly Phe
Asp Asp Asn Tyr Cys Arg Asn Pro Asp Gly Gln Pro Arg Pro Trp Cys
Tyr Thr Leu Asp Pro His Thr Arg Trp Glu Tyr Cys Ala Ile Lys Thr
Cys Ala Asp Asn Thr Met Asn Asp Thr Asp Val Pro Leu Glu Thr Thr
Glu Cys Ile Gln Gly Gln Gly Glu Gly Tyr Arg Gly Thr Val Asn Thr
Ile Trp Asn Gly Ile Pro Cys Gln Arg Trp Asp Ser Gln Tyr Pro His
Glu His Asp Met Thr Pro Glu Asn Phe Lys Cys Lys Asp Leu Arg Glu
Asn Tyr Cys Arg Asn Pro Asp Gly Ser Glu Ser Pro Trp Cys Phe Thr
Thr Asp Pro Asn Ile Arg Val Gly Tyr Cys Ser Gln Ile Pro Asn Cys
Asp Met Ser His Gly Gln Asp Cys Tyr Arg Gly Asn Gly Lys Asn Tyr
Met Gly Asn Leu Ser Gln Thr Arg Ser Gly Leu Thr Cys Ser Met Trp
Asp Lys Asn Met Glu Asp Leu His Arg His Ile Phe Trp Glu Pro Asp
Ala Ser Lys Leu Asn Glu Asn Tyr Cys Arg Asn Pro Asp Asp Asp Ala
His Gly Pro Trp Cys Tyr Thr Gly Asn Pro Leu Ile Pro Trp Asp Tyr
Cys Pro Ile Ser Arg Cys Glu Gly Asp Thr Thr Pro Thr Ile Val Asn
Leu Asp His Pro Val Ile Ser Cys Ala Lys Thr Lys Gln Leu Arg
SEQ ID NO 2:
Pro Ala Leu Pro Glu Asp Gly Gly Ser Gly Ala Phe Pro Pro Gly His
Phe Lys Asp Pro Lys Arg Leu Tyr Cys Lys Asn Gly Gly Phe Phe Leu
Arg Ile His Pro Asp Gly Arg Val Asp Gly Val Arg Glu Lys Ser Asp
Pro His Ile Lys Leu Gln Leu Gly Ile Asn Ala Glu Glu Arg Gly Val
Val Ser Ile Lys Gly Val Cys Ala Asn Arg Tyr Leu Ala Met Lys Glu
Asp Gly Arg Leu Leu Ala Ser Lys Cys Val Thr Asp Glu Cys Phe Phe
Phe Glu Arg Leu Glu Ser Asn Asn Tyr Asn Thr Tyr Arg Ser Arg Lys
Tyr Thr Ser Trp Tyr Val Ala Leu Lys Arg Thr Gly Gln Tyr Lys Leu
Gly Ser Lys Thr Gly Pro Gly Gln Lys Ala Ile Leu Phe Leu Pro Met
Ser Ala Lys Ser
SEQ ID NO 3:
Phe Asn Leu Pro Pro Gly Asn Tyr Lys Lys Pro Lys Leu Leu Tyr Cys
Ser Asn Gly Gly His Phe Leu Arg Ile Leu Pro Asp Gly Thr Val Asp
Gly Thr Arg Asp Arg Ser Asp Gln His Ile Gln Leu Gln Leu Ser Ala
Glu Ser Val Gly Glu Val Tyr Ile Lys Ser Thr Glu Thr Gly Gln Tyr
Leu Ala Met Asp Thr Asp Gly Leu Leu Tyr Gly Ser Gln Thr Pro Asn
Glu Glu Cys Leu Phe Leu Glu Arg Leu Glu Glu Asn His Tyr Asn Thr
Tyr Ile Ser Lys Lys His Ala Glu Lys Asn Trp Phe Val Gly Leu Lys
Lys Asn Gly Ser Cys Lys Arg Gly Pro Arg Thr His Tyr Gly Gln Lys
Ala Ile Leu Phe Leu Pro Leu Pro Val Ser Ser Asp
SEQ ID NO 4:
Ala Tyr Arg Pro Ser Glu Thr Leu Cys Gly Gly Glu Leu Val Asp Thr
Leu Gln Phe Val Cys Gly Asp Arg Gly Phe Tyr Phe Ser Arg Pro Ala
Ser Arg Val Ser Arg Arg Ser Arg Gly Ile Val Glu Glu Cys Cys Phe
Arg Ser Cys Asp Leu Ala Leu Leu Glu Thr Tyr Cys Ala Thr Pro Ala
Lys Ser Glu
SEQ ID NO 5:
Met Leu Lys Glu Trp Glu Leu Arg
SEQ ID NO 6:
Gly Pro Glu Thr Leu Cys Gly Ala Glu Leu Val Asp Ala Leu Gln Phe
Val Cys Gly Asp Arg Gly Phe Tyr Phe Asn Lys Pro Thr Gly Tyr Gly
Ser Ser Ser Arg Arg Ala Pro Gln Thr Gly Ile Val Asp Glu Cys Cys
Phe Arg Ser Cys Asp Leu Arg Arg Leu Glu Met Tyr Cys Ala Pro Leu
Lys Pro Ala Lys Ser Ala
SEQ ID NO 7:
Gln Ala Leu Gly Gln Asp Met Val Ser Pro Glu Ala Thr Asn Ser Ser
Ser Ser Ser Phe Ser Ser Pro Ser Ser Ala Gly Arg His Val Arg Ser
Tyr Asn His Leu Gln Gly Asp Val Arg Trp Arg Lys Leu Phe Ser Phe
Thr Lys Tyr Phe Leu Lys Ile Glu Lys Asn Gly Lys Val Ser Gly Thr
Lys Lys Glu Asn Cys Pro Tyr Ser Ile Leu Glu Ile Thr Ser Val Glu
Ile Gly Val Val Ala Val Lys Ala Ile Asn Ser Asn Tyr Tyr Leu Ala
Met Asn Lys Lys Gly Lys Leu Tyr Gly Ser Lys Glu Phe Asn Asn Asp
Cys Lys Leu Lys Glu Arg Ile Glu
SEQ ID NO 8:
Ala Leu Asp Thr Asn Tyr Cys Phe Arg Asn Leu Glu Glu Asn Cys Cys
Val Arg Pro Leu Tyr Ile Asp Phe Arg Gln Asp Leu Gly Trp Lys Trp
Val His Glu Pro Lys Gly Tyr Tyr Ala Asn Phe Cys Ser Gly Pro Cys
Pro Tyr Leu Arg Ser Ala Asp Thr Thr His Ser Thr Val Leu Gly Leu
Tyr Asn Thr Leu Asn Pro Glu Ala Ser Ala Ser Pro Cys Cys Val Pro
Gln Asp Leu Glu Pro Leu Thr Ile Leu Tyr Tyr Val Gly Arg Thr Pro
Lys Val Glu Gln Leu Ser Asn Met Val Val Lys Ser Cys Lys Cys Ser
SEQ ID NO 9:
Ser Ile Glu Glu Ala Val Pro Ala Val Cys Lys Thr Arg Thr Val Ile
Tyr Glu Ile Pro Arg Ser Gln Val Asp Pro Thr Ser Ala Asn Phe Leu
Ile Trp Pro Pro Cys Val Glu Val Lys Arg Cys Thr Gly Cys Cys Asn
Thr Ser Ser Val Lys Cys Gln Pro Ser Arg Val His His Arg Ser Val
Lys Val Ala Lys Val Glu Tyr Val Arg Lys Lys Pro Lys Leu Lys Glu
Val Gln Val Arg Leu Glu Glu His Leu Glu Cys Ala Cys Ala Thr Thr
Ser Leu Asn Pro Asp Tyr Arg Glu Glu Asp Thr Gly Arg Pro Arg Glu
Ser Gly Lys Lys Arg Lys Arg Lys Arg Leu Lys Pro Thr
SEQ ID NO 10:
Cys Asn Asp Met Thr Pro Glu Gln Met Ala Thr Asn Val Asn Cys Ser
Ser Pro Glu Arg His Thr Arg Ser Tyr Asp Tyr Met Glu Gly Gly Asp
Ile Arg Val Arg Arg Leu Phe Cys Arg Thr Gln Trp Tyr Leu Arg Ile
Asp Lys Arg Gly Lys Val Lys Gly Thr Gln Glu Met Lys Asn Asn Tyr
Asn Ile Met Glu Ile Arg Thr Val Ala Val Gly Ile Val Ala Ile Lys
Gly Val Glu Ser Glu Phe Tyr Leu Ala Met Asn Lys Glu Gly Lys Leu
Tyr Ala Lys Lys Glu Cys Asn Glu Asp Cys Asn Phe Lys Glu Leu Ile
Leu Glu Asn His Tyr Asn Thr Tyr Ala Ser Ala Lys Trp Thr His Asn
Gly Gly Glu Met Phe Val Ala Leu Asn Gln Lys Gly Ile Pro Val Arg
Gly Lys Lys Thr Lys Lys Glu Gln Lys Thr Ala His Phe Leu Pro Met
Ala Ile Thr
SEQ ID NO 11:
Asn Ser Asp Ser Glu Cys Pro Ser Ser His Asp Gly Tyr Cys Leu His
Asp Gly Val Cys Met Tyr Ile Glu Ala Leu Asp Lys Tyr Ala Cys Asn
Cys Val Val Gly Tyr Cys Gly Glu Arg Cys Gln Tyr Arg Cys Leu Lys
Trp Trp Glu Leu Arg
SEQ ID NO 12:
Glu Gly Ile Cys Arg Asn Arg Val Thr Asn Asn Val Lys Asp Val Thr
Lys Leu Val Ala Asn Leu Pro Lys Asp Tyr Met Ile Thr Leu Lys Tyr
Val Pro Gly Met Asp Val Leu Pro Ser His Cys Trp Ile Ser Glu Met
Val Val Gln Leu Ser Asp Ser Leu Thr Asp Leu Leu Asp Lys Phe Ser
Asn Ile Ser Glu Gly Leu Ser Asn Tyr Ser Ile Ile Asp Lys Leu Val
Asn Ile Val Asp Asp Leu Val Glu Cys Val Lys Glu Asn Ser Ser Lys
Asp Leu Lys Lys Ser Phe Lys Ser Pro Glu Pro Arg Leu Phe Thr Pro
Glu Glu Phe Phe Arg Ile Phe Asn Arg Ser Ile Asp Ala Phe Lys Asp
Phe Val Val Ala Ser Glu Thr Ser Asp Cys Val Val Ser Ser Thr Leu
Ser Pro Glu Lys Asp Ser Arg Val Ser Val Thr Lys Pro Phe Met Leu
Pro Pro Val Ala Ala
SEQ ID NO 13:
His Tyr Leu His Ile Arg Pro Ala Pro Ser Asp

1. A composition for topical or transdermal use having multiple recombinant human growth factors therein for repairing or regenerating human cells, keratinous materials, and/or the extracellular matrix to improve any one of skin surface appearance, cutaneous signs of chronological aging and/or aging signs induced by external factors such as prolonged exposure to ultraviolet (UV) exposure, and/or impaired surface appearance of the skin, the composition comprising:
(a) water at a concentration ranging from 55 wt % to 85 wt % of the overall composition;
(b) optionally, and when present, hyaluronic acid at a concentration ranging from 0.5 wt % to 6 wt % of the overall composition with a molecular weight ranging from 150 kDA to 600 kDA; and
(c) at least 5 but no more than 12 recombinant human growth factors selected from sh-Polypeptide-t comprising a sequence at least 90% identical to SEQ ID NO 2, sh-Polypeptide-11 comprising a sequence at least 90% identical to SEQ ID NO 3, sh-Polypeptide-31 comprising a sequence at least 90% identical to SEQ ID NO 4, shOligopeptide-2 comprising a sequence at least 90% identical to SEQ ID NO 6, sh-Polypeptide-10 comprising a sequence at least 90% identical to SEQ ID NO 7, sh-Polypeptide-5 comprising a sequence at least 90% identical to SEQ ID NO 8, sh-Polypeptide-8 comprising a sequence at least 90% identical to SEQ ID NO 9, sh-Polypeptide-3 comprising a sequence at least 90% identical to SEQ ID NO 10, sh-Polypeptide-62 comprising a sequence at least 90% identical to SEQ ID NO 1, Acetyl Octapeptide-17 Amide comprising a sequence at least 90% identical to SEQ ID NO 5, sh-Oligopeptide-1 comprising a sequence at least 90% identical to SEQ. ID NO 11, sh-Polypeptide-4 comprising a sequence at least 90% identical to SEQ ID NO 12 and Acetyl sh-Oligopeptide-77 Amide comprising a sequence at least 90% identical to SEQ ID NO 13 in which, each growth factor, when present in the composition, is present at a concentration ranging from 0.001 wt % to 1 wt % of the overall composition.

2. The composition of claim 1, wherein the composition is an aqueous solution for topical use comprising:
at least 8 growth factors are present,
excluding hyaluronic acid; and
further comprises:
(i) a nanoencapsulating agent that encapsulates the recombinant human growth factors and present in the composition at an effective amount to deliver the recombinant human growth factors to cells, keratinous materials, and/or the extracellular matrix in a human
(ii) a Swertia chirata extract present at an effective amount for stimulating endogenous keratinocyte growth factor production to induce keratinocyte proliferation and epidermis regeneration to increase skin volume and/or reduce cutaneous signs of aging,
(iii) an emollient, and
(iv) a skin protecting agent present at an effective amount to stimulate endogenous production of collagen VII, laminin-5, and/or fibronectin.

3. The composition of claim 2, wherein the Swertia chirata extract is present at a concentration ranging from 1 wt % to 8 wt % of the overall composition and the skin protecting agent is present at a concentration ranging from 1 wt % to 5 wt % of the overall composition, the skin protecting agent comprises a combination of glycerin, water, dextran, and caproyl tetrapeptide-3, and
a water soluble skin conditioning agent configured to minimize enlarged pores, tighten lax pores, improve uneven skin tone, soften fine lines and wrinkles, diminish dullness, and/or strengthen a weakened skin surface, the water soluble skin conditioning agent is present at a concentration ranging from 0.1 wt % to 5 wt % of the overall composition

4. (canceled)

5. (canceled)

6. (canceled)

7. (canceled)

8. The composition of claim 2, wherein the water soluble skin conditioning agent is niacinamide.

9. The composition of claim 8, wherein the nanoencapsulating agent is a C1-C6 alkylene glycol suitable for topical and/or transdermal use and lecithin mixture present at a ratio ranging from 4:1 to 2.3:1 of C1-C6 alkylene glycol to lecithin.

10. The composition of claim 9, wherein the C1-C6 alkylene glycol is propandiol and lecithin is a soybean (G. max) extract in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanolamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent.

11. The composition of claim 1, wherein the composition is an oil in water emulsion for topical use and transdermal absorption comprising:
at least 8 recombinant human growth factors are present, and
hyaluronic acid present at a concentration ranging from 0.5 wt % to 6 wt % of the overall composition with a molecular weight ranging from 150 kDA to 600 kDA, and
further comprising:
(i) a nanoencapsulating agent that encapsulates the recombinant human growth factors and present in an effective amount to deliver the recombinant human growth factors to cells, keratinous materials, and/or the extracellular matrix in a human;
(ii) a plurality of anti-inflammatory agents and/or antioxidants at a concentration ranging from 1 wt % to 7 wt % of the overall composition;
(iii) a combination of liposomally encapuslated bacterial derived photolyase(s), plant-derived roxisome(s), and bacterial derived endonuclease(s) that are present in an effective amount prevent and/or reduce photoaging associated with ultraviolet (UV) exposure by enhancing endogenous DNA repair.

12. The composition of claim 11, wherein the combination of liposomally encapuslated bacterial derived photolyase(s), plant-derived roxisome(s), and bacterial derived endonucleases are present at a concentration ranging from 0.3 wt % to 10 wt % of the overall composition; wherein the bacterial derived photolyases are present at a concentration ranging from 0.1 wt % to 3.5 wt % of the overall composition.

13. (canceled)

14. The composition of claim 12, wherein the bacterial derived photolyase is a cyanobacteria photolyase that is an Anacystis nidulans photolyase.

15. (canceled)

16. The composition of claim 14, wherein the plant derived roxisome(s) is 8-oxo-guanine glycosylase from A. thaliana that is present at a concentration ranging from 0.1 wt % to 3.5 wt % of the overall composition.

17. (canceled)

18. The composition of claim 16, wherein the bacterial derived endonucleases from M. Luteus present at a concentration ranging from 0.1 wt % to 3.5 wt % of the overall composition.

19. (canceled)

20. The composition of claim 11, wherein the nanoencapsulating agent is a C1-C6 alkylene glycol suitable for topical and/or transdermal use and lecithin mixture present at a ratio ranging from 4:1 to 2.3:1 of C1-C6 alkylene glycol to lecithin.

21. The composition of claim 20, wherein the C1-C6 alkylene glycol is propandiol and lecithin is a soybean (G. max) extract in which the lecithin comprises a mixture of phosphatidylcholine, phosphatidylinositol, phosphatidylethanoloamine, and phosphatidic acid in which phosphatidylcholine is present in the nanoencapsulating agent at a concentration of 14 wt % to 23 wt % of the overall concentration of the nanoencapsulating agent, phosphatidylinositol is present in the nanoencapsulating agent at a concentration 0.35 wt % to 0.7 wt % of the overall concentration of the nanoencapsulating agent, phosphatidylethanoloamine is present in the nanoencapsulating agent at a concentration of 1.0 wt % to 1.9 wt % of the overall concentration of the nanoencapsulating agent, and phosphatidic acid is present in the nanoencapsulating agent at a concentration of 0.15 wt % to 0.3 wt % of the overall concentration of the nanoencapsulating agent.

22. The composition of claim 21, further comprising an emollient and an emulsifier present at a concentration ranging from 6 wt % to 32 wt % of the overall composition: wherein the retinol is present as an antioxidant in the composition at a concentration ranging from 1 wt % to 3 wt %; wherein the liposomes encapsulating bacterial derived photolyase(s), plant-derived roxisome(s), and bacterial derived endonucleases are comprised of soybean lecithin.

23. (canceled)

24. (canceled)

25. (canceled)

26. The composition of claim 1, wherein the composition is an aqueous solution for topical use on a human scalp comprising:
at least 10 recombinant human growth factors,
the hyaluronic acid is present at a concentration ranging from 0.5 wt % to 6 wt % of the overall composition with a molecular weight ranging from 150 kDA to 600 kDA, and
a nanoencapsulating agent that encapsulates the recombinant human growth factors and present in an effective amount to deliver the recombinant human growth factors to the human scalp to induce hair growth or re-growth and/or to improve scalp appearance.

27. A kit comprising the composition of claim 1 packaged within a container and further comprising a plurality of sterile microneedles packaged within the kit and configured to enhance transdermal delivery of the composition post-application of the composition to the human cells and/or keratinous materials by creating punctures in a user's stratum corneum to induce a wound healing response and to enhance delivery to improve any one of skin surface appearance, cutaneous signs of chronological aging and/or aging signs induced by external factors such as prolonged exposure to ultraviolet (UV) exposure, and or impaired surface appearance of the skin.

28. (canceled)

29. A method of reducing wrinkles and/or fine lines over a predetermined time period, the method comprising:
(a) applying on the skin of a subject the composition of claim 1; and
(b) repeating the application of the composition to the skin of the subject at predetermined time periods thereby reducing wrinkles, fine lines, and/or the appearance thereof during the predetermined time period.

30. (canceled)

31. (canceled)

32. (canceled)

33. A method for reducing pore size over a predetermined time period, the method comprising:
(a) applying on the skin of a subject the composition of claim 1; and
(b) repeating the application of the composition to the skin of the subject at predetermined time periods thereby reducing pore size and/or the appearance thereof during the predetermined time period.

34. (canceled)

35. (canceled)

36. (canceled)

37. A method of enhancing and/or improving skin texture over a predetermined time period, the method comprising:
(a) applying on the skin of a subject the composition of claim 1; and
(b) repeating the application of the composition to the skin of the subject at predetermined time periods thereby improving and/or enhancing skin texture and/or the appearance thereof during the predetermined time period.

38. (canceled)

39. (canceled)

40. (canceled)

41. The composition of claim 1, wherein the sh-Polypeptide-1 is SEQ ID NO 2, the sh-Polypeptide-11 is SEQ ID NO 3, the sh-Polypeptide-31 is SEQ ID NO 4, the shOligopeptide-2 is SEQ ID NO 6, the sh-Polypeptide-10 is SEQ ID NO 7, the sh-Polypeptide-5 is SEQ ID NO 8, the sh-Polypeptide-8 is SEQ ID NO 9, the sh-Polypeptide-3 is SEQ ID NO 10, the sh-Polypeptide-62 is SEQ ID NO 1, Acetyl Octapeptide-17 Amide is SEQ ID NO 5, the sh-oligopeptide-1 is SEQ ID NO 11, the sh-Polypeptide-4 is SEQ ID NO 12, and the Acetyl sh-Oligopeptide-77 Amide is SEQ ID NO 13.

42. (canceled)

43. (canceled)

44. The composition of claim 1, wherein the sh-Polypeptide-1 comprises a sequence at least 98% identical to SEQ ID NO 2, the sh-Polypeptide-11 comprises a sequence at least 98% identical to SEQ ID NO 3, the sh-Polypeptide-31 comprises a sequence at least 98% identical to SEQ ID NO 4, the shOligopeptide-2 comprises a sequence at least 98% identical to SEQ ID NO 6, the sh-Polypeptide-10 comprises a sequence at least 98% identical to SEQ ID NO 7, the sh-Polypeptide-5 comprises a sequence at least 98% identical to SEQ ID NO 8, the sh-Polypeptide-8 comprises a sequence at least 98% identical to SEQ ID NO 9, the sh-Polypeptide-3 comprises a sequence at least 98% identical to SEQ ID NO 10, the sh-Polypeptide-62 comprises a sequence at least 98% identical to SEQ ID NO 1, Acetyl Octapeptide-17 Amide comprises a sequence at least 98% identical to SEQ ID NO 5, the sh-oligopeptide-1 comprises a sequence at least 98% identical to SEQ ID NO 11, the sh-Polypeptide-4 comprises a sequence at least 98% identical to SEQ ID NO 12, and the Acetyl-sh-Oligopeptide-77 Amide comprises a sequence at least 98% identical to SEQ ID NO 13.

45-60. (canceled)
